(19) United States
(12) Patent Application Publication
(10) Pub. No.: US 2025/0025284 A1
(43) Pub. Date: Jan. 23, 2025

(54) DEVICES FOR ACUTE AND CHRONIC FLOW MODIFICATION IN BODY LUMENS AND METHODS OF USE THEREOF

(71) Applicant: Nephronyx Ltd., Modiin (IL)

(72) Inventors: Sagy KARAVANY, Kibbutz Dvir (IL); Oren Moshe ROTMAN, Holon (IL); Menashe YACOBY, Shoham (IL); Tanhum FELD, Moshav Methavya (IL)

(73) Assignee: Nephronyx Ltd., Modiin (IL)

(21) Appl. No.: 18/714,554

(22) PCT Filed: Nov. 2, 2022
(86) PCT No.: PCT/IB2022/060573

§ 371 Date: May 29, 2024

Related U.S. Application Data
(60) Provisional application No. 63/264,774, filed on Dec. 1, 2021.

(51) Int. Cl. A61F 2/07; A61F 2/06

(52) U.S. Cl. CPC A61F 2/07; A61F 2002/061; A61F 2002/068; A61F 2002/072

(57) ABSTRACT

The acute and chronic devices and methods described herein include a body lumen fluid flow modulator including an upstream flow accelerator and a downstream flow decelerator. The fluid flow modulator preferably includes one or more openings that define a gap/entrainment region that provides a pathway through which additional fluid from a branch lumen(s) is entrained into the fluid stream flowing from the upstream flow accelerator to the downstream flow decelerator. The upstream flow accelerator may include a radially expandable portion that expands responsive to upstream pressures within the body lumen to accommodate a wider range of fluid flow rates. Additionally, the flow modulator may include a plurality of collapsible fixation elements for securing the flow modulator within the body lumen.

### CROSS-REFERENCE TO RELATED APPLICATIONS

This application claims priority to U.S. Provisional Patent Application No. 63/264,774, filed Dec. 1, 2021, the entire contents of which are incorporated herein by reference.

### FIELD OF THE INVENTION

The present invention relates generally to acute and chronic devices and methods for modifying flow in body lumens, such as devices and methods for creating pressure differences and/or entrainment of fluid at lumens that branch off from other lumens for enhancing fluid flow to treat different disorders or diseases.

### BACKGROUND OF THE INVENTION

Heart failure is the physiological state in which cardiac output is insufficient to meet the needs of the body and the lungs. Patients suffering from any of a number of forms of heart failure are prone to increased fluid in the body. Congestive heart failure (CHF) occurs when cardiac output is relatively low and the body becomes congested with fluid. There are many possible underlying causes of CHF, including myocardial infarction, coronary artery disease, valvular disease, and myocarditis. Chronic heart failure is associated with neurohormonal activation and alterations in autonomic control. Although these compensatory neurohormonal mechanisms provide valuable support for the heart under normal physiological circumstances, they also have a fundamental role in the development and subsequent progression of CHF. For example, one of the body's main compensatory mechanisms for reduced blood flow in CHF is to increase the amount of salt and water retained by the kidneys. Retaining salt and water, instead of excreting it into the urine, increases the volume of blood in the bloodstream and helps to maintain blood pressure. However, the larger volume of blood also stretches the heart muscle, enlarging the heart chambers, particularly the ventricles. At a certain amount of stretching, the heart's contractions become weakened, and the heart failure worsens. Another compensatory mechanism is vasoconstriction of the arterial system. This mechanism, like salt and water retention, raises the blood pressure to help maintain adequate perfusion.

Glomerular filtration rate (GFR), the rate at which the kidney filters blood, is commonly used to quantify kidney function and, consequently, the extent of kidney disease in a patient. Individuals with normal kidney function exhibit a GFR of at least 90 mL/min with no evidence of kidney damage. The progression of kidney disease is indicated by declining GFR, wherein a GFR below 15 mL/min generally indicates that the patient has end stage renal disease (ESRD), which is the complete failure of the kidney to remove wastes or concentrate urine.

In addition to increases in total body salt and water, it has also been found that altered capacitance of the splanchnic venous vessels change the blood volume distribution. Decreased venous capacitance can lead to shifts of fluid from the venous reservoir into the effective circulatory volume/splanchnic circulation, thus increasing filling pressures. This could result in clinical heart congestion.

Cardiovascular problems, such as but not limited to, inadequate blood flow or chronic hypertension, may lead to fluid retention in the kidneys, chronic kidney disease, lowered GFR, renal failure or even ESRD. For example, hypertension is considered the second most prevalent cause for kidney failure (after diabetes). It has been estimated that hypertension causes nephrotic damage and lowers GFR.

Transjugular intrahepatic portosystemic shunt (TIPS or TIPSS) is an artificial channel within the liver that establishes communication between the inflow portal vein and the outflow hepatic vein. Generally, under imaging guidance, a small metal stent is placed to keep the channel open and allow the channel to bring blood draining from the bowel back to the heart while avoiding the liver. TIPS may be used to treat conditions such as portal hypertension (often due to liver cirrhosis) which frequently leads to intestinal bleeding, life-threatening esophageal bleeding (esophageal varices), and the buildup of fluid within the abdomen (ascites), and has shown promise for treating hepatorenal syndrome. A drawback of TIPS is that blood meant to be filtered by the liver bypasses the liver via the artificial channel, which may cause complications.

Therefore, it would be desirable to provide acute and/or chronic apparatus and methods to improve blood flow to prevent disease, improve body functionality, and/or treat conditions that would benefit from modified body fluid flow. For example, it would be desirable to treat heart failure, treat hypertension, prevent kidney disease, improve kidney functionality, restore normal values of splanchnic circulation, improve liver functionality, enhance or replace TIPS, and/or prevent blood clots from flowing through vasculature to sensitive portions of the body, such as the brain, in order to prevent strokes.

It would further be desirable to provide anchoring mechanisms for securing a flow modulator device within the body lumen.

Additionally, it would be desirable to provide a flow modulator device with efficacy across a wide range of blood flow.

### SUMMARY OF THE INVENTION

The present invention seeks to provide acute and chronic devices and methods for altering flow in body lumens. For example, devices and methods are provided for creating pressure differences and/or fluid entrainment at lumens that branch off from other lumens for enhancing or modifying fluid flow to treat different disorders or diseases. For positioning, the device may be acutely or chronically implanted within the body lumen.

The devices and methods of the present invention have many applications. For example, the device may be used to reduce pressure and improve flow, thereby improving flow in stenotic body lumens. It also may be used in the aortic arch to reduce peak systolic pressure in the brain or divert emboli to other portions of the body (e.g., the legs) and thereby reduce the risk of stroke. The device further may be installed in a bifurcation (e.g., in the brachiocephalic vessels) to reduce peak pressure gradients or to divert emboli with very little energy loss.

The devices and methods of the present invention have particular application in treating blood flow to and from the kidneys. In accordance with one embodiment, the device is configured to be installed near one of the renal arteries or in the inferior vena cava near the branch off to the renal veins or in one of the renal veins. When installed in the inferior vena cava or in the renal vein, the device can create (due to the Bernoulli effect or other factors) a region in the inferior vena cava or in the renal vein which has increased blood velocity and reduced pressure. In this manner, blood may be drawn from the kidneys to the renal veins and then to the inferior vena cava, thereby improving kidney functionality and reducing necrotic damage to the kidneys.

When installed in or near the renal vein, the devices of the present invention may improve renal function by improving net filtration pressure, which is glomerular capillary blood pressure−(plasma-colloid osmotic pressure+Bowman's capsule hydrostatic pressure), e.g., 55 mm Hg−(30 mm Hg+15 mm Hg)=10 mm Hg. The devices and methods of the present invention thus provide an improvement over existing therapies, such as diuretics (although the invention can be used in addition to diuretics), angiotensin-converting enzyme inhibitors (ACEIs), and angiotensin receptor blockers (ARBs), which can have deleterious effects on kidney function. When used in conjunction with current modes of treatment such as diuretics, the devices and methods of the present invention are expected to improve the response for diuretics and reduce the dosage needed to obtain therapeutic benefit of such previously known therapies, without the disadvantages of these existing therapies.

The devices and methods of the present invention may be used to divert flow from the kidneys to the inferior vena cava with little energy loss. For example, with a small energy loss due to pressure drop and other fluid factors, a significantly greater increase in blood flow may be achieved. This diversion of flow from the kidneys with little energy loss to increase blood flow is expected to treat conditions such as heart failure and/or hypertension.

It is noted that there is a significant difference between use of an upstream nozzle with no downstream flow decelerator, such as a diffuser. If only an upstream nozzle is placed in the flow path, there is significant energy loss downstream of the nozzle due to the sudden expansion of flow. However, by using a downstream flow decelerator, such as a diffuser, the energy loss is significantly reduced. This leads to another advantage: since the energy loss is significantly reduced, the additional flow that flows into the gap is efficiently added to the flow from the upstream flow accelerator.

In addition, the present invention is expected to provide optimal structure for an upstream flow accelerator when used together with a downstream flow decelerator. For example, the distance between the outlet of the upstream flow accelerator and the inlet of the downstream flow decelerator should be less than a predetermined length to reduce pressure at the gap between the outlet and the inlet.

When installed in the renal artery, the device can reduce pressure applied to the kidneys. Without being limited by any theory, high blood pressure can cause damage to the blood vessels and filters in the kidney, making removal of waste from the body difficult. By reducing the pressure in the renal artery, the filtration rate improves. Although there may be a reduction in the perfusion pressure, the filtration rate will increase because the overall kidney function is more efficient.

It is noted that the fluid flow modulator of the present invention may modulate fluid flow without any input from an external energy source, such as a fan, motor, and the like and without any moving parts. The structure of the device of the invention transfers energy from one lumen flow to another different lumen flow with minimal flow energy losses. Some of the energy loss (in the form of pressure loss and/or reduced volume) may beneficially reduce heart preload (i.e., the filling pressure). For example, in healthy patients, the Frank-Starling mechanism dictates that increases in preload will increase cardiac output; however, in the setting of impaired contractility (as is the case with many chronic and acute heart failure patients), this relation is reversed such that preload increases can cause a reduction in cardiac output. Thus, it is desirable to reduce preload in heart failure patients in order to improve cardiac function. It is especially desirable to reduce preload in heart failure patients with reduced renal blood flow as reduced as reduced renal blood flow leads to fluid accumulation which increases both filling pressure and renal congestion.

In accordance with one aspect of the present invention, a flow modulator device is provided for altering fluid flow through a body lumen coupled to a branch lumen. The flow modulator device may include a stent sized and shaped to be positioned within the body lumen. The stent may include an upstream component having an inlet, an outlet, and a cross-sectional flow area that converges from the inlet towards the outlet to form a nozzle, at least a portion of the nozzle being radially expandable responsive to pressure upstream of the flow modulator device within the body lumen, a downstream component having an entry, an exit, and a cross-sectional flow area that diverges from the entry towards the exit, and an entrainment region between the inlet of the upstream component and the exit of the downstream component. The entrainment region may include one or more openings, e.g., a plurality of openings radially spaced around the entrainment region. Accordingly, the nozzle accelerates a fluid stream passing through the upstream component towards the downstream component to generate a low pressure region in a vicinity of the entrainment region that entrains additional fluid into the fluid stream via the one or more openings as the fluid stream passes into the downstream component.

The upstream component and the downstream component may be formed from a frame, and may be at least partially coated with a biocompatible material, thereby exposing the one or more openings and defining the inlet. In one embodiment, the radially expandable portion of the nozzle is formed by a continuous passageway of biocompatible material extending through the flow modulator device within an uncoated portion of the frame. For example, the continuous passageway of biocompatible material may have an outer diameter less than an inner diameter of the uncoated portion of the frame, such that the continuous passageway of biocompatible material radially expands toward the uncoated portion of the frame responsive to pressure upstream of the flow modulator device within the body lumen.

In another embodiment, the radially expandable portion of the nozzle may be formed by a coated portion of the frame having more flexibility than other portions of the frame, such that it radially expands responsive to pressure upstream of the flow modulator device within the body lumen. For example, the coated portion of the frame having more flexibility than other portions of the frame may be thin Nitinol wires. Moreover, the biocompatible material coating the portion of the frame having more flexibility than other portions of the frame may radially expand beyond an outer diameter of the frame responsive to pressure upstream of the flow modulator device within the body lumen.

In another embodiment, the radially expandable portion of the nozzle may be formed by a continuous passageway of biocompatible material extending along a frameless portion of the flow modulator device between the frame forming at least a portion of the upstream component and the frame forming the downstream component, such that the continuous passageway of biocompatible material radially expands responsive to pressure upstream of the flow modulator device within the body lumen. In yet another embodiment, the radially expandable portion of the nozzle may be formed by a portion of the frame comprising a first, straight portion extending from the downstream component toward the inlet of the upstream component, and a second portion extending circumferentially and axially from the first, straight portion toward the inlet of the upstream component, such that the portion of the frame radially expands responsive to pressure upstream of the flow modulator device within the body lumen.

The frame may define a plurality of cells. The plurality of cells may be adjacent to the inlet of the upstream component or the exit of the downstream component. The plurality of cells may comprise an oval shape. At least some of the cells may comprise a preformed bend configured to extend outwardly to secure the flow modulator device within the body lumen.

In addition, at least a portion of a distal portion of the downstream component may not be coated with the biocompatible material, such that the distal portion may adapt with the body lumen to thereby prevent migration of the flow modulator device within the body lumen. Moreover, the radially expandable portion of the nozzle may radially expand responsive to pressure upstream of the flow modulator device within the body lumen without causing expansion of portions of the upstream and downstream components that engage the body lumen. The stent may be transitionable from a collapsed delivery state to an expanded deployed state within the body lumen.

In addition, at least one of the upstream component or the downstream component may include a plurality of fixation elements extending outwardly therefrom to secure flow modulator device within the body lumen. For example, the plurality of fixation elements may include a first portion that extends outwardly in a downstream direction, and a second portion that extends from the first portion outwardly in an upstream direction. The plurality of fixation elements may be transitionable from a collapsed delivery state to an expanded deployed state.

Moreover, the upstream component may include a retrieval portion for facilitating retrieval of the flow modulator. For example, the retrieval portion may include a constricted section at an upstream end of the flow modulator, such that the retrieval portion converges from the inlet towards the upstream end. Additionally, the retrieval portion may include a hook at the constricted section at the upstream end of the flow modulator. The hook may be pulled to collapse the upstream component. In some embodiments, the retrieval portion includes a plurality of wires extending from the inlet to the constricted section, each wire of the plurality of wires having a length that is longer than a radius of the inlet, and shorter than a diameter of the inlet.

The downstream component may include a first diverging portion and a second diverging portion downstream from the first diverging portion, such that the second diverging portion's average angle of divergence greater than the first diverging portion's average angle of divergence. Moreover, the downstream component may be a diffuser that decelerates the fluid stream having the entrained additional fluid passing through the downstream component. The entrainment region may be integrally formed with the downstream component.

Moreover, the downstream component may include a retrieval portion for facilitating retrieval of the flow modulator. For example, the retrieval portion may include a constricted section at a downstream end of the flow modulator, such that the retrieval portion converges from the exit towards the downstream end. Additionally, the retrieval portion may include a hook at the constricted section at the upstream end of the flow modulator. The hook may be pulled to collapse the downstream component. In some embodiments, the retrieval portion includes a plurality of wires extending from the exit to the constricted section, each wire of the plurality of wires having a length that is longer than a radius of the exit, and shorter than a diameter of the exit.

In some embodiments, the flow modulator device may have a delivery system including a delivery catheter having one or more holders. The one or more holders may be releasably coupled to the flow modulator device when the flow modulator device is in a collapsed delivery state. The delivery system may also include a sheath, which may be configured to be disposed over the delivery catheter and the flow modulator device to maintain the flow modulator device in a collapsed delivery state. In embodiments, the sheath may be retracted relative to the delivery catheter to expose the flow modulator device, such that the flow modulator device may transition to an expanded deployed state.

The one or more holders may include a stationary component coupled to the delivery catheter, and a sliding component slidably coupled to the stationary component and comprising at least one knob configured to secure the flow modulator device to the sliding component. The sliding component may passively slide relative to the stationary component in response to force applied to the sliding component by the flow modulator device.

In accordance with another aspect of the present invention, the flow modulator device may include a stent sized and shaped to be positioned within the body lumen. The stent may include an upstream component having an inlet, an outlet, and a cross-sectional flow area that converges from the inlet towards the outlet, a downstream component having an entry, an exit, and a cross-sectional flow area that diverges from the entry towards the exit, and an entrainment region between the inlet of the upstream component and the exit of the downstream component. The entrainment region may include one or more openings. The flow modulator device may accelerate a fluid stream passing through the upstream component towards the downstream component to generate a low pressure region in a vicinity of the entrainment region that entrains additional fluid into the fluid stream via the one or more openings as the fluid stream passes into the downstream component.

In some embodiments, the flow modulator may include a plurality of fixation elements extending outwardly from at least one of the upstream component or the downstream component to secure the flow modulator device within the body lumen. The first portion may extend outwardly in a downstream direction, and the second portion may extend outwardly in an upstream direction. In another embodiment, the plurality of fixation elements may be coupled to the frame via an attachment portion. The plurality of fixation elements may also include an anchor portion, where the anchor portion may flip over the attachment portion between an upstream extended configuration and a downstream extended configuration.

In another embodiment, the flow modulator device may include a retrieval portion extending from the downstream component, where the retrieval portion may facilitate retrieval of the flow modulator device. The retrieval portion may include a constricted section at a downstream end of the flow modulator device, such that the retrieval portion converges from the exit towards the downstream end. In further embodiments, the retrieval portion may have a hook at the constricted section at the downstream end of the flow modulator device, the hook configured to be pulled to collapse the downstream component. In additional embodiments, the flow modulator device may also include an upstream retrieval portion extending from the upstream component, where the upstream retrieval portion may facilitate retrieval of the flow modulator device. The upstream retrieval portion may include a constricted section at an upstream end of the flow modulator device, such that the retrieval portion converges from the inlet towards the upstream end.

The upstream component and the downstream component may be at least partially coated with a biocompatible material, thereby exposing the one or more openings and defining the inlet. For example, at least a portion of a proximal portion of the upstream component may not be coated with the biocompatible material. The proximal portion may therefore adapt within the body lumen, thereby preventing migration of the flow modulator device within the body lumen.

In some embodiments, the flow modulator may include an upstream retrieval portion extending from the upstream component to facilitate retrieval of the flow modulator device. The upstream retrieval portion may include a constricted section at an upstream end of the flow modulator device, such that the retrieval portion converges from the inlet towards the upstream end. The upstream component and the downstream component may at least be partially coated with a biocompatible material, thereby exposing the one or more openings and defining the inlet. Moreover, at least a portion of a proximal portion of the upstream component may not be coated with the biocompatible material, such that the proximal portion may adapt with the body lumen to thereby prevent migration of the flow modulator device within the body lumen.

### BRIEF DESCRIPTION OF THE DRAWINGS

FIG. 1 is a side view of an exemplary fluid flow modulator constructed in accordance with the principles of the present invention.

FIG. 2A illustrates the frame structure of the fluid flow modulator of FIG. 1.

FIG. 2B illustrates the frame structure of FIG. 2A having selectively coated portions of biocompatible material in accordance with the principles of the present invention.

FIG. 2C is a close up view of an eyelet of the frame structure of FIG. 2B.

FIG. 2D illustrates a hook coupled to the eyelet of FIG. 2C in accordance with the principles of the present invention.

FIGS. 3A to 3C illustrate alternative eyelets of the frame structure constructed in accordance with the principles of the present invention. FIG. 3D illustrates a hook coupled to the alternative eyelet of FIGS. 3A to 3C in accordance with the principles of the present invention.

FIGS. 4A to 4C illustrate an exemplary fluid flow modulator having an alternative retrieval portion constructed in accordance with the principles of the present invention.

FIGS. 5A and 5B illustrate another exemplary fluid flow modulator having an alternative retrieval portion constructed in accordance with the principles of the present invention.

FIGS. 6A to 6F illustrate alternative exemplary fluid flow modulators constructed in accordance with the principles of the present invention.

FIG. 7 illustrates an exemplary fluid flow modulator having an expandable nozzle in accordance with the principles of the present invention.

FIGS. 8A and 8B illustrate an exemplary expandable nozzle constructed in accordance with the principles of the present invention.

FIG. 9A illustrates a fluid flow modulator having an alternative exemplary expandable nozzle constructed in accordance with the principles of the present invention, FIG. 9B illustrates the frame structure of the fluid flow modulator of FIG. 9A, and FIG. 9C is a cross-sectional view of the fluid flow modulator of FIG. 9A.

FIG. 10A illustrates a fluid flow modulator having another alternative exemplary expandable nozzle constructed in accordance with the principles of the present invention, FIG. 10B illustrates the frame structure of the fluid flow modulator of FIG. 10A, and FIG. 10C is a cross-sectional view of the fluid flow modulator of FIG. 10A.

FIG. 11A illustrates another alternative exemplary expandable nozzle constructed in accordance with the principles of the present invention, FIG. 11B illustrates a back view of the expandable nozzle of FIG. 11A, and FIGS. 11C and 11D illustrate the expansion of the expandable nozzle of FIG. 11A.

FIG. 12A illustrates a fluid flow modulator having exemplary fixation elements constructed in accordance with the principles of the present invention, FIG. 12B illustrates the fixation element of FIG. 12A, and FIG. 12C illustrates the frame structure of the fluid flow modulator of FIG. 12A.

FIG. 12D illustrates deployment of the fixation elements of the fluid flow modulator of FIG. 12A.

FIGS. 13A to 13D illustrate various alternative fixation elements of the fluid flow modulator constructed in accordance with the principles of the present invention.

FIGS. 14A to 14C illustrate alternative fixation elements of the fluid flow modulator constructed in accordance with the principles of the present invention.

FIGS. 15A illustrates deployment of the fixation elements of the fluid flow modulator of FIGS. 14A-14C, and FIGS. 15B to 15D illustrates retrieval of the fluid flow modulator of FIGS. 14A to 14C.

FIGS. 16 to 16F illustration alternative fixation elements of the fluid flow modulator constructed in accordance with the principles of the present invention.

FIGS. 17A and 17B illustrate a delivery system for an alternative fluid flow modulator constructed in accordance with the principles of the present invention.

FIG. 17C illustrates an alternative delivery system for the fluid flow modulator of FIGS. 17A and 17B.

FIGS. 18A to 18C illustrate a slidable fluid flow modulator holder mechanism constructed in accordance with the principles of the present invention

FIGS. 18D to 18F illustrate deployment of a fluid flow modulator using the slidable fluid flow modulator holder mechanism of FIGS. 18A to 18C.

### DETAILED DESCRIPTION OF EMBODIMENTS

Devices and methods for altering flow in body lumens are provided for creating pressure differences and/or to induce fluid entrainment from branch lumens for enhancing or modifying fluid flow to treat different disorders or diseases.

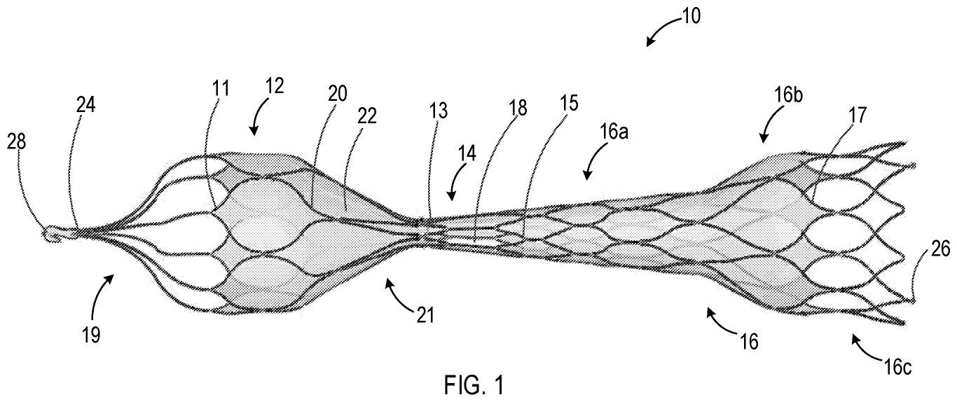

Referring to FIG. 1, flow modulator 10 constructed and operative in accordance with the principles of the present invention is provided. Specifically, FIG. 1 is a side view of flow modulator 10 having upstream component 12, downstream component 16, and an entrainment region, e.g., gap 14, disposed between upstream component 12 and downstream component 16. The entrainment region may be integrally formed in downstream component 16 or in upstream component 12, or both. Gap 14 is designed to entrain fluid into a stream of fluid flowing from upstream component 12 to downstream component 16. As described below, upstream component 12 and downstream component 16 create a lower pressure region in the vicinity of gap 14, which preferably entrains fluid into the stream of fluid flowing across gap 14. Fluid entrainment is induced by shear-induced turbulent flux. In accordance with the principles of the invention, such entrainment is expected to transport blood or other body fluids to or from a region so as to improve organ function (e.g., from the renal vein(s) to the inferior vena cava to promote better functionality of the kidney(s) and/or from the hepatic vein(s) to the inferior vena cava to improve liver function, thereby treating disorders and/or diseases such as heart failure).

Upstream component 12 has inlet 11 and outlet 13, and has a cross-sectional flow area that converges in a downstream direction, e.g., from upstream component 12 towards downstream component 16, along part or all of the length of upstream component 12, thereby forming a nozzle. In this manner, upstream component 12 accelerates flow of fluid through upstream component 12. Downstream component 16 has entry 15 and exit 17, and has a cross-sectional flow area that diverges in a downstream direction along part or all of the length of downstream component 16, thereby forming a diffuser. As shown in FIG. 1, downstream component 16 may include first diverging portion 16a, second diverging portion 16b downstream from first diverging portion 16a, and uncovered portion 16c downstream from second diverging portion 16b. The average angle of divergence of second diverging portion 16b preferably is greater than the average angle of divergence of first diverging portion 16a.

Uncovered portion 16c may be less rigid than the sealing zone of downstream component 16, and may adapt to the vessel without damaging the vessel, thereby preventing migration of flow modulator 10 during, e.g., coughing or other events that may cause a dramatic change in vessel diameter. Downstream component 16 thus decelerates flow of fluid through downstream component 16. The distance between outlet 13 and entry 15, e.g., the length of gap 14, is selected to generate a low pressure region in the vicinity of gap 14, while minimizing pressure loss and reducing resistance to fluid flow from the branch lumen(s), e.g., renal flow.

PCT International Patent Application Publications WO 2016/128983, WO 2018/029688, WO 2018/220589, WO 2019/097424, and WO 2020/109979, and U.S. Pat. No. 10,195,406 describe several converging and diverging structures that may be utilized as the flow modulator in accordance with the principles described herein, and the disclosures of each of those patents/applications are incorporated herein by reference in their entireties. Other converging and diverging structures suitable for use in accordance with the principles of the present invention are described herein. In addition, the present invention may be implemented using other kinds of converging and diverging structures, such as Stratford ramp nozzles (e.g., in which flow through the nozzle is on the verge of separation, which gives the diffuser the best length to efficiency ratio), de Laval nozzles (e.g., asymmetric hourglass shape), variable cross-sectional area nozzles and venturis, ramped nozzles and venturis, and others.

The central axis of the diverging portion may be disposed in-line with, or offset from, the central axis of the converging portion. As shown in FIG. 1, upstream component 12 and downstream component 16 share common, collinear flow axis. Alternatively, upstream component 12 may be angled with respect to downstream component 16. Upstream component 12 and downstream component 16 also may lie along a continuously curved path.

Upstream component 12 and downstream component 16 may be constructed as grafts, stents (coated or uncoated), stent grafts (coated or uncoated), and the like, and are formed of biocompatible materials, such as stainless steel or Nitinol. The outer contours of any of upstream component 12 and downstream component 16 may be sealed against the inner wall of the body lumen (such as by being expanded thereagainst), or alternatively may not be sealed, depending on the particular application. This may be referred to as the fixation area(s).

In accordance with one aspect of the present invention, flow modulator 10 is sized and shaped to be implanted in a body lumen. Flow modulator 10 may be compressed for delivery (e.g., percutaneous delivery within a delivery sheath) and expanded upon deployment (e.g., self-expanding upon release from the end of the delivery sheath or balloon expandable), as described in U.S. Pat. No. 11,324,619 to Yacoby, the entire contents of which is incorporated by reference in its entirety herein. Flow modulator 10 may be inserted into the body lumen in an antegrade or retrograde manner and similarly may be removed antegrade or retrograde. Flow modulator 10 may be used as an acute device to be removed after few hours/days or a chronic permanent device or a device that can be retrieved after long-term implantation. Additionally, flow modulator 10 may be decoupled from the delivery device and left in the patient for, e.g., 1-5 days or preferably 3 days, before retrieval and removal from the patient's body. When used as an acute device, flow modulator 10 may remain coupled to a delivery/retrieval device, e.g., sheath and/or wire/shaft, throughout the short-term implantation for ease of device delivery and retrieval. Flow modulator 10 may be compressible while disposed within a body lumen to allow periodic wash-out of stagnant flow zones created adjacent to flow modulator 10. For example, flow modulator 10 may be partially or fully reduced in diameter within the body lumen to allow blood flow through a stagnant flow zone.

Preferably, upon expansion, flow modulator 10 is sized to contact the inner wall of the body lumen to anchor flow modulator 10 within the lumen. Specifically, upstream component 12 may have a fixation area sized for anchoring upstream component 12 within the body lumen in its expanded, deployed state. For example, the fixation area of upstream component 12 may be sized to contact the inner wall of the body lumen and preferably has a diameter the size of, or slightly larger than, the diameter of the body lumen. The fixation area of upstream component 12 may have a constant diameter for a length suitable for anchoring upstream component 12 in the body lumen. Similarly, downstream component 16 may have a fixation area sized for anchoring downstream component 16 within another portion of the body lumen. For example, the fixation area of downstream component 16 may include at least a portion of second diverging portion 16b and/or uncovered portion 16c of downstream component 16. The fixation area of downstream component 16 may be sized to contact the inner wall of the other portion of the body lumen and preferably has a diameter the size of, or slightly larger than, the diameter of that portion of the body lumen. The fixation area of downstream component 16 may have a constant diameter for a length suitable for anchoring downstream component 16 in the body lumen. Preferably the fixation areas of upstream component 12 and downstream component 16 are configured to seal fluid modulator 10 within the body lumen so that fluid only flows into the fluid channels created by fluid modulator 10 and does not flow between the fixation areas of upstream component 12 and downstream component 16 and the vessel wall.

Flow modulator 10 may be formed from one or more frames and may be coated with one or more biocompatible materials. For example, the frame(s) may be formed of a metal (e.g., shape memory metal) or alloy or a combination thereof (e.g., a stent made of stainless steel or Nitinol or cobalt chromium). For some applications, the frame(s) may include a braided stent. In the case of more than one frame, the frames may be joined together by a suitable technique, such as welding. For example, upstream component 12 and downstream component 16 may be formed from a common frame or two frames that may be joined prior to implantation.

Flow modulator 10 may be constructed from frame 20 forming a plurality of cells, and frame 20 of flow modulator 10 may be at least partially coated with biocompatible material 22. As shown in FIG. 1, flow modulator 10 may be only partially covered with biocompatible material 22 such that a plurality of cells upstream of inlet 13, a plurality of cells forming uncovered portion 16c, and a plurality of cells at gap 14 remain uncoated. Specifically, upstream component 12 may be coated with biocompatible material 22 to define the fluid flow channel through upstream component 12, such that fluid flowing through a body lumen enters inlet 11, accelerates through the converging portion of upstream component 12, and exits out outlet 13 into the entrainment region of fluid modulator 10 having gap 14. A low pressure region is formed at gap 14 by the shapes of upstream component 12 and downstream component 16. Additional fluid from the branch lumen(s) at gap 14 is entrained into the fluid stream passing from outlet 13 to entry 15, via plurality of openings 18 formed by the uncoated portions at gap 14. Downstream component 16 also may be coated with biocompatible material 22 to define the fluid flow channel through downstream component 16, e.g., first diverging portion 16A and second diverging portion 16B, such that the fluid stream from outlet 13 together with the additional fluid passing through plurality of openings 18 at gap 14 enter entry 15, decelerate through the diverging portion of downstream component 16, and exit out exit 17 back into the body lumen, e.g., across uncovered portion 16c, which remains uncoated as described in further detail below.

Biocompatible material 22 may be a fabric and/or polymer such as expanded polytetrafluoroethylene (ePTFE), woven, knitted, and/or braided polyester, polyurethane, DACRON (polyethylene terephthalate), silicone, polycarbonate urethane, or pericardial tissue from an equine, bovine, or porcine source. The biocompatible coating may impede or block fluid flow where applied to the frame. The order of the joining and coating processes may be joining before coating or coating before joining. Biocompatible material 22 may be coupled to the frame(s) via stitching, spray coating, encapsulation, electrospinning, dip molding, and/or a different technique. In some embodiments, biocompatible material 22 may be expandable, at least along some portions of flow modulator 10, to thereby adjust the cross-sectional area of the flow path through flow modulator 10 responsive to the pressure gradient across flow modulator 10, as described in further detail below.

Alternatively, flow modulator 10 may be coated with a hydrophilic, hemocompatible coating (active such as heparin coating or passive) or a drug coating. In addition, flow modulator 10 may be selectively coated in different areas. For example, flow modulator 10 may include a drug coating on the sealing zones (the portions of flow modulator 10 that contact tissue) to prevent tissue adhesion to the IVC wall, and a heparin coating on the portions of flow modulator 10 where there is constant contact with blood to thereby prevent thrombus formation.

In a preferred embodiment, biocompatible material 22 is fluid impermeable. However, for some applications, the surfaces need not be impermeable, but may have a permeability that is sufficiently low as to substantially prevent blood from flowing through the longitudinal portion of the body lumen via any flow path other than through the flow channel defined by the inner surfaces of flow modulator 10. For some applications, each of the surfaces has permeability per unit length of less than 0.25 micrometers (e.g., between 0 and 0.25 micrometers), where the permeability per unit length is defined based upon the following equation, which is based upon Darcy's Law: k/Δx=Vμ/Δp, where k is permeability, Δx is length (in meters), V is average velocity (in meters per second), μ is fluid viscosity (measured in Pascal-seconds), and ΔP is the pressure differential measured in Pascals).

Although the invention is not bound by any theory, a simplified engineering explanation is now provided to help understand how upstream component 12 and downstream component 16 operate to create reduced pressure at gap 14.

The Bernoulli equation governs the relationship between fluid velocity and pressure (neglecting the height difference):

$${P}_{1} + \frac{1}{2} \cdot ρ \cdot {V}_{1}^{2} = {P}_{2} + \frac{1}{2} \cdot ρ \cdot {V}_{2}^{2} + {E}_{loss}$$

P=pressure
ρ=density
V=velocity
1=conditions at the inlet (upstream component 12)
2=conditions at gap 14
Mass conservation (same flow rate):
V₁·A₁=V₂·A₂
A=Flow cross section
Eₗₒₛₛ=Energy loss

For example, if flow modulator 10 is installed near the kidneys with upstream component 12 in the inferior vena cava, then V₁ and A₁ are the velocity and flow area, respectively, at the inferior vena cava.

The flow velocity at the gap (V₂) is designed to achieve the desired pressure reduction. For example, for 0.5 meter per second velocity and 3 times area ratio, a suction of about 6-8 mm Hg can be achieved. In the case of deployment near the kidney, this pressure differential is expected to improve renal function by improving renal perfusion pressure. The pressure will change due to improvement in the renal flow.

Applicant has discovered that using a maximum distance between the outlet of the upstream component and the entry to the downstream component will improve flow rates in the branched vessel(s) with relatively low pressure loss. A distance too great will create a significant pressure loss that actually sends flow in the wrong direction in the renal vein(s). In addition, other structural characteristics of the downstream component improve renal flow with low pressure loss such as a greater inner diameter at the entry of the downstream component than the inner diameter at the outlet of the upstream component, a greater length of the diverging area of the downstream component than the length of the converging area of the upstream component, and/or a lesser average angle of divergence of the downstream component than the average angle of convergence of the upstream component.

In another example, flow modulator 10 may be installed near a bifurcation to divert emboli from the bifurcation. In yet another example, flow modulator 10 may be deployed in the aortic arch to reduce peak systolic pressure.

The dimensions of flow modulator 10 may be suitable for implantation in the inferior vena cava. In particular, inlet 11 of upstream component 12 may be configured to be disposed upstream from a branch off to a renal vein(s), downstream component 16 may be configured to be disposed in the inferior vena cava, such that exit 17 is downstream from the branch off to the renal vein(s), and gap 14 may be disposed in the vicinity of the branch to the renal vein(s). Accordingly, the diameter of inlet 11 in the deployed, expanded state may range from 12-40 mm. The diameter of outlet 13 of upstream component 12 may be selected to create a jet velocity for a given device resistance. In the example of chronic cases, the diameter of outlet 13 may range from 3.5-8 mm. In acute cases, the diameter of outlet 13 preferably ranges from 3-7 mm. Moreover, flow modulator 10 may have an outer diameter at its upstream and downstream sealing zones ranging from 15 to 50 mm, and preferably 20 to 40 mm, and an overall length between 100-200 mm, and preferably 150 mm. In some embodiments, the outer diameter at the downstream sealing zone may be larger than the outer diameter at the upstream sealing zone. For example, the outer diameter at the downstream sealing zone may be 30 to 40 mm, e.g., 36 mm, and the outer diameter at the upstream sealing zone may be 20 to 30 mm, e.g., 28 mm.

The length of the fixation area of upstream component 12 may range from 5-30 mm. The overall length of upstream component 12 may range from 15-60 mm. In accordance with the principles of the present invention, a shorter distance from outlet 13 of upstream component 12 to entry 15 of downstream component 16 will provide better performance for downstream component 16, but will result in lower renal flow because there is a greater resistance to flow from the renal vein(s) to downstream component 16. Thus, the distance from outlet 13 to entry 15 preferably is selected (e.g., in a range from −5-25 mm) to provide improved renal flow rate with minimal pressure loss.

The distance from outlet 13 of upstream component 12 to a center line of the branched lumen, e.g., the right renal vein, and may range from −25 mm to 100 mm. The length of the fixation area of downstream component 16 may range from 5-30 mm. The overall length of downstream component 16 is preferably greater than the overall length of upstream component 12 because a diverging shape creates a much higher pressure loss than a converging shape. For example, the length of first diverging portion 16A alone may be greater than the length of upstream component 12. The ratio of the overall length of upstream component 12 and the overall length of downstream component 16 may range from 1:1 to 3:1. The diameter at entry 15 of downstream component 16 is preferably larger than the diameter at outlet 13 of upstream component 12. Thus, the cross-sectional flow area at outlet 13 of upstream component 12 is less than the cross-sectional flow area at entry 15 of downstream component 16. The diameter at entry 15 of downstream component 16 is selected to receive all the fluid jetted from outlet 13. The ratio of the diameter at entry 15 of downstream component 16 and the diameter at outlet 13 of upstream component 12 may range from 1:1 to 2:1. In addition, the diameter at entry 15 of downstream component 16 may be greater when the distance between outlet 13 and entry 15 is larger to ensure receipt of the fluid jetted from upstream component 12. The diameter of exit 17 in the deployed, expanded state and may range from 12-40 mm.

In some embodiments, the cross-sectional flow area at outlet 13 of upstream component 12 may be expandable, e.g., responsive to the pressure gradient across flow modulator 10, to thereby adjust the cross-sectional area of the flow path of the nozzle portion of flow modulator 10, as described in further detail below. For example, during an ambulatory activity such as walking, the higher flow, and accordingly, higher upstream pressure, may cause the nozzle portion of the flow modulator to expand to thereby reduce the pressure loss. Accordingly, the ratio of the diameter at entry 15 of downstream component 16 and the diameter at outlet 13 of upstream component 12 may vary during the patient's therapy.

Moreover, the average angle of divergence in downstream component 16 and may range from 5-30 degrees. Preferably, the angle of divergence in downstream component 16 is less than the angle of convergence in upstream component 12, and is expected to prevent pressure loss. In addition, downstream component 16 should have slow change in area adjacent to entry 15, e.g., closer to the renal vein, as any additional pressure loss will reduce the inferior vena cava flow rate and thus will reduce the effectiveness of the device. The angle of divergence in downstream component 16 may be constant or may change along the length of downstream component 16. When the angle of divergence changes along the length, the angle of divergence is preferably smallest (e.g., in a range from 5-30 degrees) adjacent to entry 15. A slow change in the cross-sectional flow area adjacent to entry 15 is preferred because the fluid velocity decreases as the cross-sectional flow area increases, hence the pressure loss. Accordingly, the angle of divergence is smallest at entry 15 where the fluid flow is at maximum velocity within downstream component 16.

As shown in FIG. 1, flow modulator 10 may include retrieval portion 19 at the proximal end of upstream component 12, configured to facilitate retrieval of flow modulator 10. Retrieval portion 19 may include constricted section 24 at an upstream end of flow modulator 10. Constricted section 24 allows flow modulator 10 to remain coupled to a delivery system. In the expanded, deployed state, the cross-sectional area of retrieval portion 19 converges from inlet 11 to constricted portion 24, where retrieval portion 19 is coupled together near the center of the flow path. Retrieval portion 19 preferably is uncoated such that a fluid stream flows across the retrieval portion 19 and through inlet 11 into upstream component 12. Moreover, uncoated retrieval portion 19 may optionally serve as a filter, e.g., against thrombus and/or emboli in blood. As described above, the overall length of downstream component 16 is preferably greater than the overall length of upstream component 12 (not including retrieval portion 19). Thus, the length from inlet 11 to outlet 13 of upstream component 12 may be less than the length from entry 15 to exit 17 of downstream component 16.

As shown in FIG. 1, a retrieval device, e.g., hook 28, may be coupled to constricted portion 24 to pull retrieval portion 19 towards a delivery sheath to compress flow modulator 10 into the delivery sheath for retrieval as described in further detail below. Hook 28 may be coupled to constricted portion 24 as a separate component that is, e.g., molded, glued, compressed, welded, etc. to frame 20. In this manner a retriever, e.g., a hook or goose-neck snare device, may be coupled to hook 28 to pull retrieval portion 19 towards a delivery sheath to compress flow modulator 10 into the sheath for retrieval. Additionally, hook 28 may be pulled in a direction(s) away from gap 14 to partially or fully reduce the diameter of flow modulator 10 within a body lumen. Such reduction would allow for wash-out of any stagnant flow zones created adjacent to flow modulator 10. Flow modulator 10 could then be fully removed, repositioned within the body lumen and expanded, or expanded in the prior deployment location within the body lumen. As described in further detail below, additionally or alternatively, flow modulator 10 may include a retrieval portion at the distal end of downstream component 16 to facilitate retrieval of flow modulator 10, e.g., via the jugular vein.

In accordance with one aspect of the present invention, the downstream-most portion of downstream component 16 may form an atraumatic end of flow modulator 10 to prevent vessel damage and flare out during device crimping, and to give the distal end integrity. In the expanded, deployed state, the atraumatic end curves inward away from the body vessel inner wall. Accordingly, even after downstream component 16 is in its expanded, deployed state, flow modulator 10 may be readjusted within the body lumen with a reduced risk of injury to the vessel wall of the body lumen due to the distal end of flow modulator 10. In this embodiment, the cells formed by the frame of flow modulator 10 adjacent to the atraumatic end preferably is uncoated as shown in FIG. 1, such that the fluid stream flows out through exit 17 of downstream component 16 and across the uncoated, bare-metal frame of the atraumatic tip without additional acceleration due to a convergence of the flow path.

In addition, flow modulator 10 may include plurality of anchors 26 radially spaced around a downstream end of downstream component 16. Plurality of anchors 26 are configured to be coupled to a delivery device to maintain downstream component 26 in a collapsed delivery state upon exposure to a body lumen from a sheath of the delivery device to facilitate readjustment of flow modulator 10 within the body lumen. In addition, plurality of anchors 26 may function as a downstream component retrieval portion in addition to the retrieval portion of upstream component 12, such that flow modulator 10 may be retrieved from the jugular.

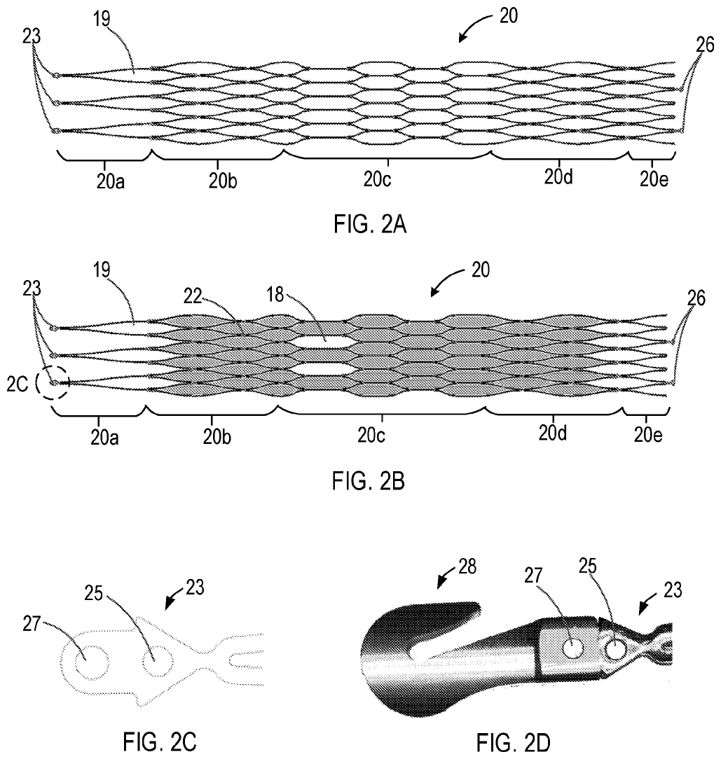

Referring now to FIG. 2A, flow modulator 10 may be formed from a single frame structure 20. FIG. 2A illustrates frame 20 as cut and flattened to show the frame cutting pattern. Illustratively, upstream component 12 and downstream component 16 are defined by frame 20. Frame 20 is preferably formed from a metal tube that is laser cut to define a plurality of cells and then processed (e.g., heated) to form the shape of flow modulator 10. Retrieval portion 19 is illustratively formed from first plurality of cells 20a, e.g., with no junctions from eyelets 23 to the sealing zone of upstream component 12 to thereby prevent flow disruption. For example, frame 20 may include straight struts extending from eyelets 23 toward inlet 11 without any junctions therebetween. Upstream component 12 is illustratively formed from second plurality of cells 20b. Downstream component 16 is formed from two different configurations of cells. First diverging portion 16a of downstream component 16 may be formed from third plurality of cells 20c and second diverging portion 16b may be formed from fourth plurality of cells 20d. Third plurality of cells 20c preferably is disposed between second plurality of cells 20b and fourth plurality of cells 20d. Uncovered portion 16c may be formed from fifth plurality of cells 20e.

The void space may be the area of the cell defined by the struts of the frame. For example, the struts may define close-looped shapes therewithin, such as ellipses or diamonds or a combination thereof. The cells of plurality of cells 20b, 20c, 20d may be constructed as described in WO 2020/109979, the entire contents of which is incorporated by reference in its entirety herein. For example, the average void space area of second plurality of cells 20b may be larger than the average void space area of third plurality of cells 20c, and may be substantially identical to the average void space area of fourth plurality of cells 20d to create a more flexible structure than third plurality of cells 20c. Thus, frame 20 may be a three-part stent forming a flexible/rigid/flexible configuration. In addition, third plurality of cells 20c may include larger, yet more rigid cell shapes (e.g., elongated hexagonal shaped cells), and second plurality of cells 20b and fourth plurality of cells 20d may include smaller, yet more flexible cell shapes (e.g., diamond shaped cells).

Advantageously, after implantation, the flexible regions can change in diameter responsive to changes in vessel diameter while the more rigid portion of the stent structure remains constant. For example, the maximum outer diameter of upstream component 12 and downstream component 16 may change in diameter responsive to changes in vessel diameter while the shape of the outlet of the nozzle of upstream component 12 and/or the intermediate section (e.g., first diverging portion 16a) of flow modulator 10 does not change. In this manner, the angle of divergence of first diverging portion 16a may remain constant even though the size of the vessel changes. The change in diameter in the vessel may be measured with, for example, one or more sensors on flow modulator 10, e.g., at the sealing zones of upstream component 12 and downstream component 16, imaging guidance such as fluoroscopy, ultrasound for evaluating the diameter change over time, and/or other external transmitters for measuring other derived parameters that may be used to measure the diameter change over time. Additionally, one or more sensors and/or imaging guidance may be used to measure the diameter of the nozzle over time.

As an additional or alternative way to enhance rigidity of the intermediate section of flow modulator 10, the struts of frame 20 at the intermediate section may be wider and/or thicker than the struts of frame 20 at the more flexible portions. For example, the struts of frame 20 may be wider and/or thicker at the section forming third plurality of cells 20c than at the sections forming second plurality of cells 20b and/or fourth plurality of cells 20d. Additionally or alternatively, the lengths of the cells formed by the struts of frame 20 may be shortened and/or the number of cells for a given length of frame 20 may be decreased to increase rigidity.

In accordance with another aspect of the present invention, the relative flexibility between the portions of the frame may be selected using different shaped cells, e.g., diamond shape or hexagonal shape. For example, the flexibility of second plurality of cells 20b and fourth plurality of cells 20d, and the rigidity of third plurality of cells 20c, may be selected based on the shape of the respective void space defined by the struts of frame 20 (e.g., flexibly-shaped cells/rigidly-shaped cells/flexibly-shaped cells in the frame). In addition, the cells having larger overall void space area may be stronger than the cells having a larger overall working area. Accordingly, the plurality of cells defining gap 14, e.g., third plurality of cells 20c, may have an overall larger average void space area while maintaining desired rigidity, such that a gap may be formed larger within a respective cell, thereby increasing the amount of flow that can be entrained through the gap than could be through a gap within a smaller diamond shaped cell.

As shown in FIG. 2B, frame 20 may be at least partially coated with biocompatible material 22 denoted by the shaded void space areas, to thereby define inlet 11, exit 17, and plurality of openings 18. As described above, plurality of openings 18 at gap 14 may be defined by the uncoated plurality of cells between upstream component 12 and downstream component 16. For example, frame 20 of flow modulator 10 may initially be entirely coated with biocompatible material 22, and then selected portions of the coating may be removed, e.g., via cutting, melting, laser, chemical, etc., to form gap 14 and/or uncovered portion 16c. Accordingly, at least a portion of frame 20 forming gap 14 and/or uncovered portion 16c may remain partially coated with biocompatible material 22 after selected portions of the coating are removed. Alternatively, the frame of flow modulator 10 may be selectively coated such that portions that are not coated define plurality of openings 18 at gap 14 and/or uncovered portion 16c during the coating process. Accordingly, openings 18 and/or uncovered portion 16c may not be coated during the coating process. As shown in FIG. 2B, plurality of openings 18 may include uncoated portions along a single row of cells that define a plurality of longitudinally extending openings radially spaced around the entrainment region. Moreover, a pattern of the plurality of uncoated cells of gap 14 forming plurality of openings 18 may be selected to improve entrainment properties of fluid through gap 14 when in use in a blood vessel.

Fifth plurality of cells 20e are shaped such that uncovered portion 16c is more flexible than second diverging portion 16b, e.g., the sealing zone of downstream component 16. Accordingly, uncovered portion 16c may adapt to the vessel without damaging the vessel, e.g., when the vessel is small, and further prevent migration of flow modulator 10 during, e.g., coughing or other events that may cause a dramatic change in vessel diameter.

As shown in FIGS. 2A and 2B, the distal end of fifth plurality of cells 20e may include one or more anchors 26 for assisting in maintaining downstream component 16 in its collapsed, delivery state upon exposure from a delivery sheath, as described in further detail below. In accordance with another aspect of the present invention, frame 20 does not include anchors 26, and the uncoated, bare-metal portion of fifth plurality of cells 20e forming the distal end of downstream component 16 may be used to maintain downstream component 16 in its collapsed, delivery state upon exposure from the delivery sheath.

As shown in FIGS. 2A and 2B, retrieval portion 19 of upstream component 12 may include one or more eyelets 23, e.g., pushers or pullers, to facilitate deployment and/or retrieval of flow modulator 10 from a compressed state within the delivery sheath to an expanded state outside of the delivery sheath when force is exerted on hook 28. FIG. 2C is a close-up view of eyelet 23 of FIG. 2B. As shown in FIG. 2C, eyelet 23 may include hole 25 for facilitating attachment of hook 28 to eyelet 23. Hole 25 may subsequently be filled or intentionally left empty during operation. In addition, eyelet 23 may include pin 27 sized and shaped to engage with a corresponding hole of hook 28 for securing hook 28 to eyelet 23, e.g., via welding, glue, riveting, or press fit, as shown in FIG. 2D. As shown in FIG. 2D, the angled surface of eyelet 23 may provide a smooth transition at the engagement point between hook 28 and eyelet 23, thereby preventing a retrieval device, e.g., a snare, from being caught therebetween. In the expanded, deployed state, the retrieval portion of upstream component 12 may converge from inlet 11 towards constricted section 24, and thus, one or more eyelets 23 may meet together at constricted section 24 where they may be coupled to hook 28. Accordingly, when retrieval portion 19 of upstream component 12 includes more than one eyelet, all the eyelets may be coupled to hook 28.

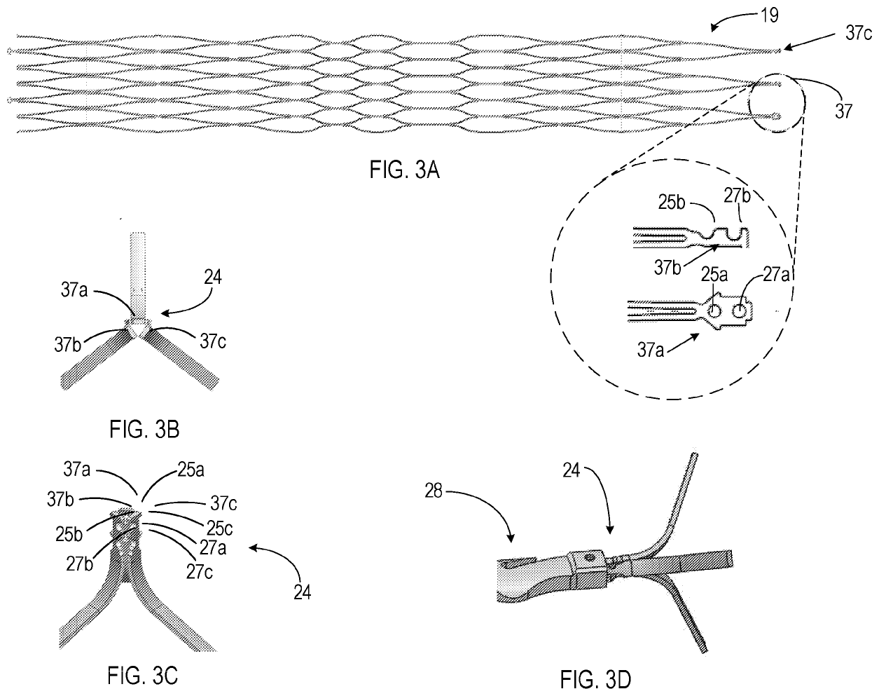

Referring now to FIGS. 3A to 3D, an alternative exemplary retrieval portion of the flow modulator is provided. As shown in FIGS. 3A to 3C, retrieval portion 19 of upstream component 12 may include one or more eyelets 37, e.g., pushers or pullers, to facilitate deployment and/or retrieval of flow modulator 10 from a compressed state within the delivery sheath to an expanded state outside of the delivery sheath when force is exerted on hook 28. As shown in FIG. 3A, retrieval portion 19 may include three eyelets, e.g., 37a, 37b, 37c. Eyelet 37a may be constructed similar to eyelet 23 of FIG. 2C, and may include hole 25a for facilitating attachment of hook 28 to eyelet 37. Hole 25a may subsequently be filled or intentionally left empty during operation. In addition, eyelet 37a may include hole 27a sized and shaped to receive a pin for securing hook 28. Eyelets 37b, 37c may include semi-circle shaped holes 25b, 25c for facilitating attachment of hook 28 to eyelet 37. In addition, eyelets 37b, 37c may include semi-circle shaped holes 27b, 27c sized and shaped to receive a pin for securing hook 28.

As shown in FIGS. 3B to 3C, eyelets 37a, 37b, 37c may converge from inlet 11 toward constricted section 24, and thus, eyelets 37a, 37b, 37c may meet together at constricted section 24 where they may be coupled to hook 28. Eyelets 37a, 37b, 37c may converge such that constricted section 24 has a triangular cross-section. As shown in FIG. 3C, eyelets 37a, 37b, 37c may converge such that semi-circle shaped holes 25b, 25c are aligned and form an opening that is aligned with hole 25a, and semi-circle shaped holes 27b, 27c are aligned and form an opening that is aligned with hole 27a. Accordingly, hook 28 may be configured to receive constricted section 24, such that a pin may be inserted through hole 27a and through the opening formed by semi-circle shaped holes 27b, 27c, to thereby secure hook 28 to eyelet 37. The opening formed by semi-circle shaped holes 25b, 25c may subsequently be filled or intentionally left empty during operation. For example, hook 28 may be secured to eyelet 37 with the pin, e.g., via welding, glue, riveting, or press fit, as shown in FIG. 3D. This arrangement of eyelets 37a, 37b, 37c may be effective to decrease the overall length of flow modulator 10, which may be advantageous in certain circumstances, e.g., in treating certain patients.

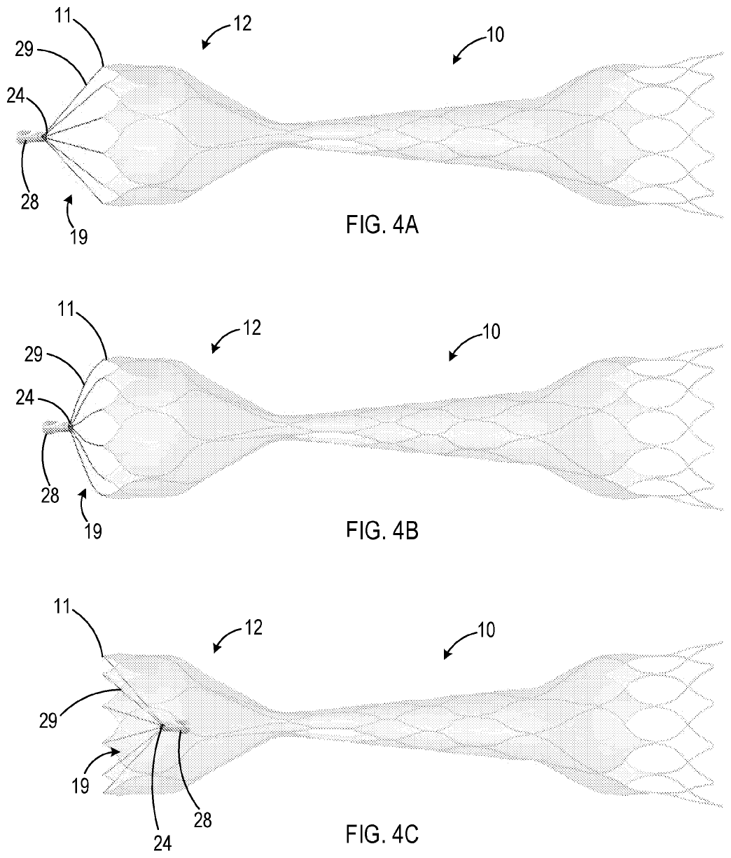

Referring now to FIGS. 4A to 4C, an alternative exemplary retrieval portion of the flow modulator is provided. As shown in FIGS. 4A to 4C, retrieval portion 19 of flow modulator 10 may be formed of plurality of wires 29, e.g., polymeric surgical wire. Wires 29 may be fixed at one end to the frame forming inlet 11 of upstream component 12, and at the other end to constricted section 24. As shown in FIG. 4A, wires 29 may extend from inlet 11 and converge toward constricted section 24, where wires 29 may be coupled to hook 28, which may be pulled to collapsed upstream component 12 via wires 29 and facilitate retrieval of flow modulator 10. FIG. 4A illustrates wires 29 fully extended proximally from upstream component 12, without collapsing upstream component 12, and FIG. 4B illustrates wires 29 loosely extended proximally from upstream component 12.

Each of wires 29 may have the same length. For example, each of wires 29 may have a length that is longer than the radius of inlet 11, and shorter than the diameter of inlet 11. Accordingly, wires 29 are sufficiently long enough to extend proximally from upstream component 12, as shown in FIGS. 4A and 4B, as well as distally within upstream component 12, as shown in FIG. 4C, while being sufficiently short enough such that hook 28 cannot reach or come into contact with the frame forming upstream component 12.

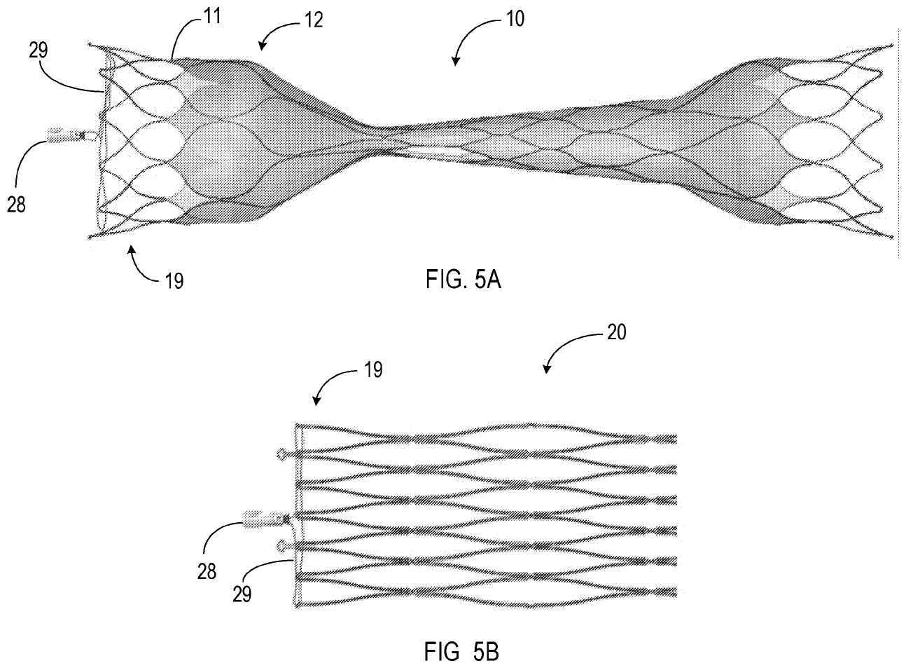

Referring now to FIGS. 5A to 5B, an alternative exemplary retrieval portion of the flow modulator is provided. As shown in FIGS. 5A to 5B, retrieval portion 19 of flow modulator 10 may be formed by wire 29, e.g., polymeric surgical wire. Wire 29 may be looped around and/or through the frame forming the upstream end of upstream component 12, e.g., wire 29 may loosely encircle the circumference of the frame at the upstream end. Moreover, wire 29 may be looped around and/or through the upstream end of upstream component 12 in a manner such that the free ends of wire 29 are coupled to hook 28 at the center point of the frame forming the upstream end, e.g., at a point equidistance from the circumference of flow modulator 10 at the upstream end. Accordingly, hook 28 may be pulled to collapsed upstream component 12 via wires 29 and facilitate retrieval of flow modulator 10.

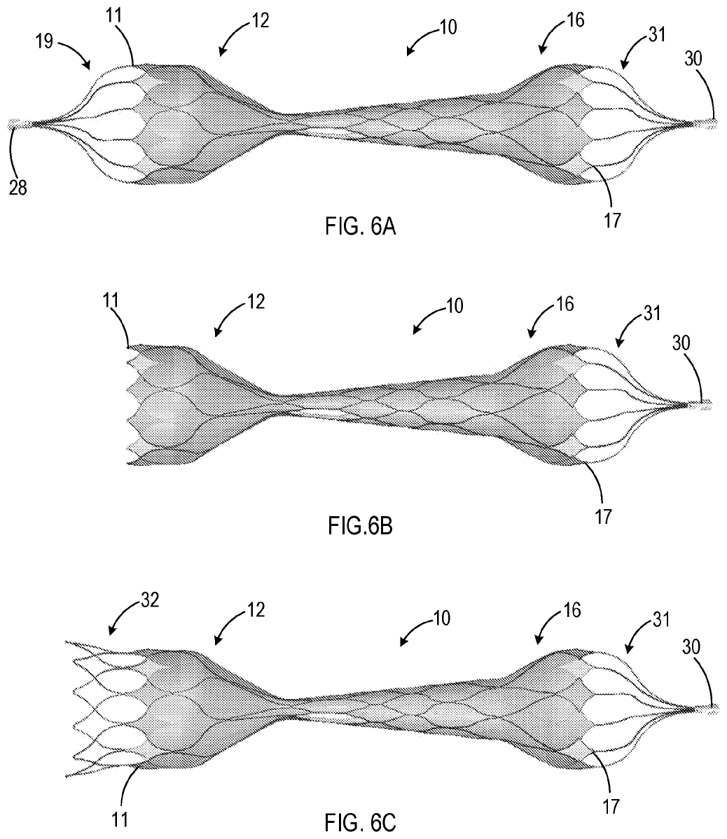

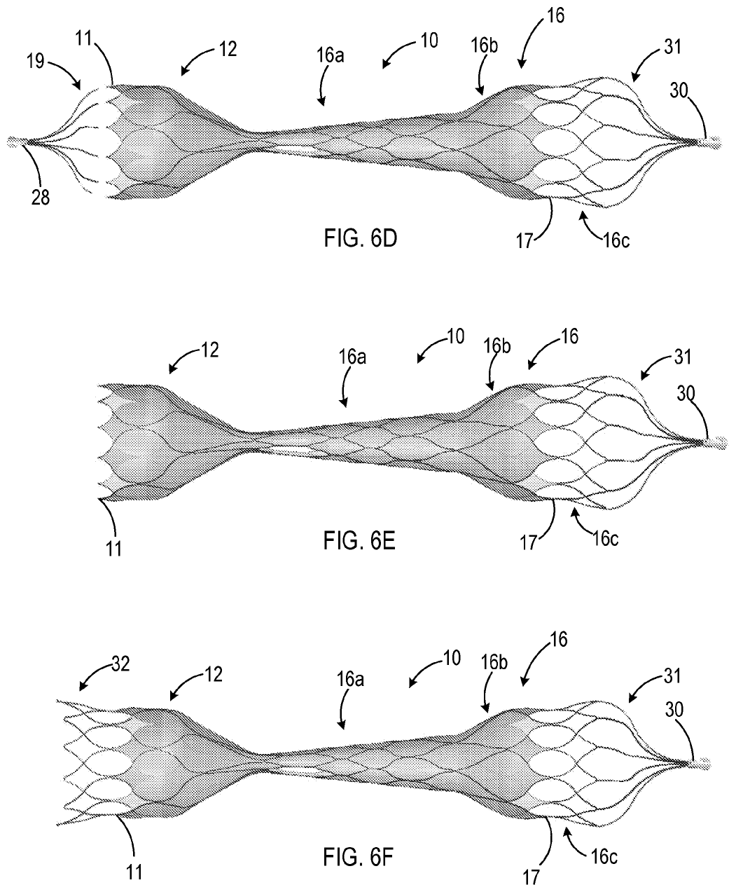

Referring now to FIGS. 6A to 6F, alternative exemplary fluid flow modulators are provided. As shown in FIG. 6A, flow modulator 10 may be constructed similar to the flow modulator of FIG. 1, except instead of uncovered portion 16c extending distally from exit 17 of downstream component 16, flow modulator 10 may include downstream retrieval portion 31 extending distally from exit 17 in the downstream direction. Downstream retrieval portion 31 may be constructed similar to upstream retrieval portion 19, such that retrieval portion 31 extends from exit 17 and converges toward a constricted section having hook 30, which may be pulled distally to collapse downstream component 16 to facilitate retrieval of flow modulator 10, e.g., via the jugular. As shown in FIG. 6A, flow modulator 10 may include both upstream retrieval portion 19 for retrieval of flow modulator 10 via the femoral vein, and downstream retrieval portion 31 for retrieval of flow modulator 10 via the jugular vein.

As shown in FIG. 6B, flow modulator 10 may be constructed similar to the flow modulator of FIG. 6A, except flow modulator 10 does not have upstream retrieval portion 19 extending from inlet 11. Instead, the upstream-most portion of upstream component 12 may form an atraumatic end of flow modulator 10 to prevent vessel damage. In the expanded, deployed state, the atraumatic end may curve inward away from the body vessel inner wall. Accordingly, even after upstream component 12 is in its expanded, deployed state, flow modulator 10 may be readjusted within the body lumen, e.g., via hook 30 and downstream retrieval portion 31, with a reduced risk of injury to the vessel wall of the body lumen due to the proximal end of flow modulator 10.

As shown in FIG. 6C, flow modulator 10 may be constructed similar to the flow modulator of FIG. 6B, except flow modulator 10 may include uncoated portion 32 extending proximally from inlet 11 in the upstream direction. Uncovered portion 32 may be less rigid than the sealing zone of upstream component 12, and may adapt to the vessel without damaging the vessel, thereby preventing upstream migration of flow modulator 10 during, e.g., coughing or other events that may cause a dramatic change in vessel diameter.

Referring now to FIGS. 6D to 6F, the flow modulator 10 may be constructed similar to the flow modulator of FIG. 6A to 6C, respectively, except flow modulator 10 may include uncovered portion 16c positioned between exit 17 of downstream component 16 and downstream retrieval portion 31. Uncovered portion 16c may be less rigid than the sealing zone of downstream component 16, and may adapt to the vessel without damaging the vessel, thereby preventing migration of flow modulator 10 during, e.g., coughing or other events that may cause a dramatic change in vessel diameter. As will be understood by a person having ordinary skill in the art, retrieval portions 19 and/or 31 of FIGS. 6A to 6F may be constructed similar to the retrieval portion of FIGS. 4A to 4C, having plurality of wires 29.

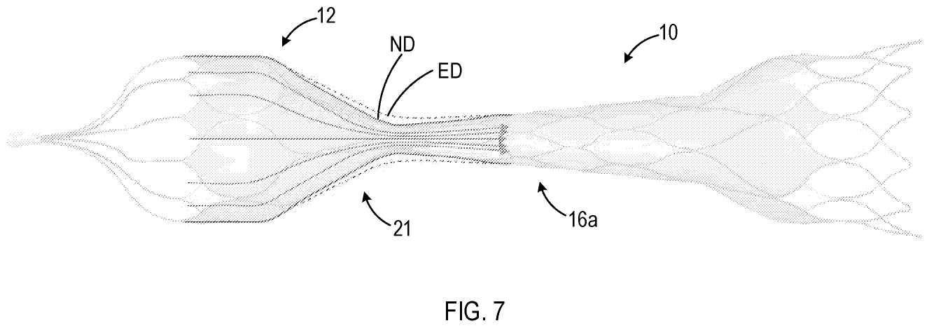

Referring now to FIG. 7, an exemplary fluid flow modulator having an expandable nozzle is provided. As shown in FIG. 7, the nozzle portion of flow modulator 10 formed at least in part by upstream component 12 may include expandable portion 21. Expandable portion 21 may have a diameter that changes responsive to a pressure gradient within the body lumen upstream of flow modulator 10. For example, as upstream IVC flow (denoted by the arrows in FIG. 7) increases, the upstream pressure increases as well, thereby creating a pressure gradient across flow modulator 10, which may cause the diameter of expandable portion 21 to adjust and increase toward extended diameter ED in response to the increased upstream pressure, which increases the cross-sectional area of the flow path of the nozzle. Moreover, when upstream IVC flow, and accordingly upstream pressure, decreases, the diameter of expandable portion 21 may adjust and decrease back toward its nominal diameter ND.

Accordingly, the dynamic characteristics of expandable portion 21 permits the smallest diameter of the nozzle to increase and decrease responsive to pressure upstream of flow modulator 10, such that the efficacy of flow modulator 10 may be extended to a wider range of IVC flows, e.g., from very low to very high IVC flows, without causing high pressure gradients during high IVC flow rates. Additionally, expandable portion 21 may expand and contract independent to the diameters of the upstream and downstream sealing zones of flow modulator 10, e.g., the portions of upstream component 12 and downstream component 16 that engage with the inner wall of the body lumen. Thus, changes in nozzle diameter does not impose changes to the stent diameter at the sealing zones.

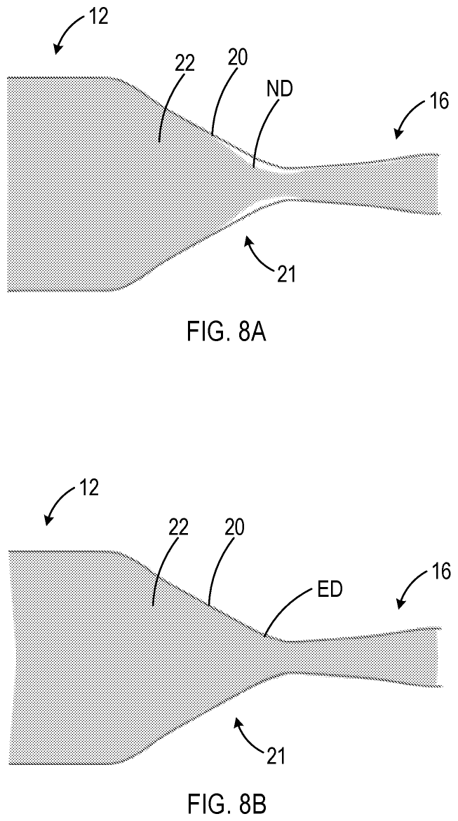

Referring now to FIGS. 8A and 8B, an exemplary expandable portion of the nozzle of the dynamic flow modulator is provided. Expandable portion 21 may be formed by a section of biocompatible material 22 extending within and unattached to frame 20 along the longitudinal axis of flow modulator 10, thereby forming a continuous passageway within frame 20 at expandable portion 21. For example, the frame forming the proximal portion of upstream component 12 and the frame forming downstream portion 16 may be coated with biocompatible material 22, whereas a portion of the frame forming expandable portion 21 between the coated proximal portion of upstream component 12 and the coated portion of downstream component 16 may remain uncoated. As shown in FIG. 8A, biocompatible material 22 may coat the frame forming the proximal portion of upstream component 12, and then extend continuously within and unattached from the frame forming expandable portion 21 until it coats downstream component 16, e.g., upstream of the entrainment region of the flow modulator. Therefore, biocompatible material 22 will form a continuous fluid flow path from upstream component 12, and through expandable portion 21 toward downstream component 16. Moreover, the unattached portion of biocompatible material 22 will have a diameter, e.g., nominal diameter ND, that is less than the diameter of frame 20, e.g., expanded diameter ED, at expandable portion 21. Accordingly, as upstream pressure increases, the unattached portion of biocompatible material 22 may expand radially from nominal diameter ND towards expanded diameter ED, thereby increasing the cross-sectional area of the flow path of the nozzle.

As described above, biocompatible material 22 may be coupled to frame via, e.g., stitching, spray coating, encapsulation, electrospinning, dip molding, and/or a different technique. For example, a tube of biocompatible material 22 may be positioned within the lumen of frame 20, and an upstream mandrel may be positioned within the tube of biocompatible material 22 within the portion of upstream component 12 to be coated, and a downstream mandrel may be positioned within the tube of biocompatible material 22 within the portion of downstream component 16 to be coated, such that a portion of the tube of biocompatible material 22 adjacent to the portion of frame 20 forming expandable portion 21 does not have a mandrel therewithin. Accordingly, the portions of the tube of biocompatible material 22 sandwiched between the mandrels and frame 20 may be heated to be coupled to frame 20, thereby leaving a continuous passageway of biocompatible material 22 extending within and unattached to frame 20 at expandable portion 21. As will be understood by a person having ordinary skill in the art, other techniques may be used to couple biocompatible material 22 to frame 22 to form expandable portion 21.

FIG. 8A illustrates nominal diameter ND of biocompatible material 22 at expandable portion 21, e.g., when expandable portion 21 is not expanded due to increased upstream pressures. As described above, expandable portion 21 may expand radially responsive to increased upstream pressures towards expanded diameter ED, e.g., the diameter of frame 20, as shown in FIG. 8B. Frame 20 may prevent biocompatible material 22 from expanding beyond the diameter of frame 20.

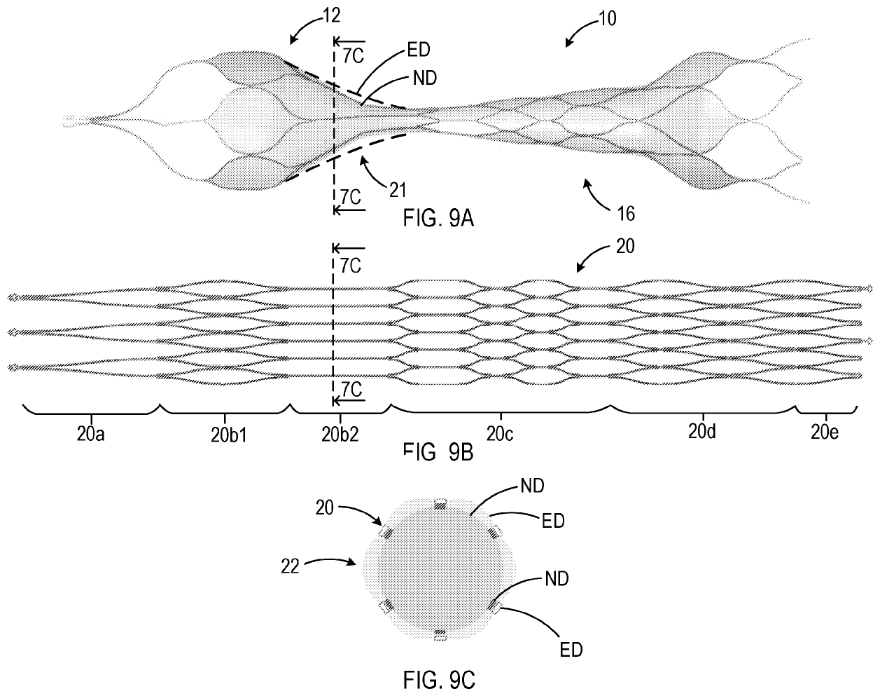

Referring now to FIGS. 9A to 9C, another exemplary expandable portion of the nozzle of the dynamic flow modulator is provided. As shown in FIGS. 9A and 9B, the portion of frame 20 forming expandable portion 21 may be formed of a plurality of thin, straight struts, e.g., thin Nitinol wires, which have more flexibility that the other portions of frame 22 that form the proximal portion of upstream component 12 and downstream component 16. For example, as shown in FIG. 9B, the plurality of thin struts may extend from the frame forming the proximal portion of upstream component 12, e.g., cells 20b1, to the frame forming downstream portion 16, e.g., cells 20c, thereby forming cells 20b2 and providing a continuous overall stent frame. The coated frame forming cells 20b2 forms expandable portion 21. Accordingly, the plurality of thin struts allow the portion of frame 20 forming expandable portion 21 to expand radially responsive to increased upstream pressures. Moreover, biocompatible material 22 coating the thin struts at expandable portion 21 also may be radially expandable responsive to increased upstream pressures.

FIG. 9C is a cross-sectional view of the nozzle portion along line 7C-7C of FIGS. 9A and 9B. As shown in FIG. 9C, the thin struts of frame 20 forming expandable portion 21 may expand radially from nominal diameter ND towards expanded diameter ED, and biocompatible material 22 at expandable portion 21 may expand radially from nominal diameter ND towards expanded diameter ED. As further shown in FIG. 9C, biocompatible material 22 may expand radially beyond the outer diameter of frame 20, to thereby accommodate even high upstream pressures.

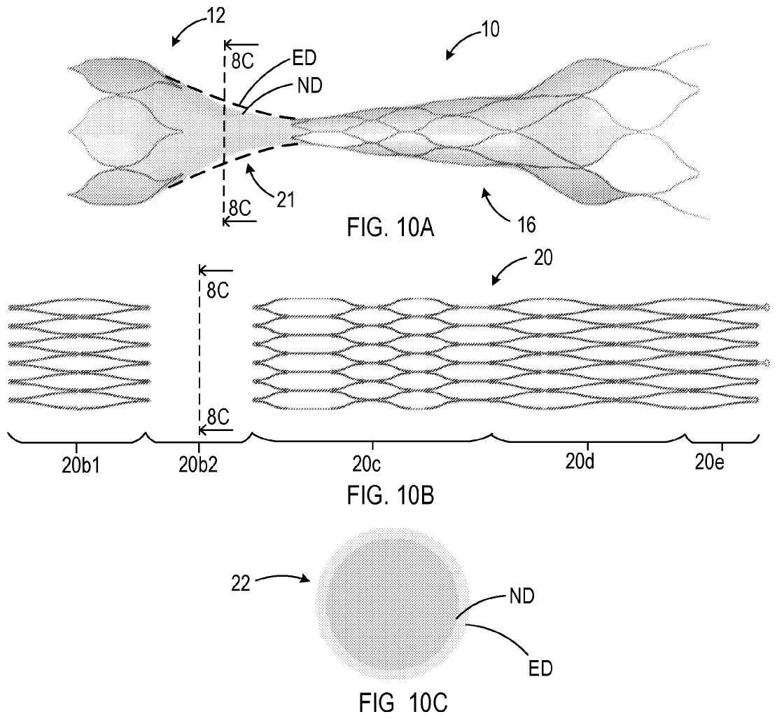

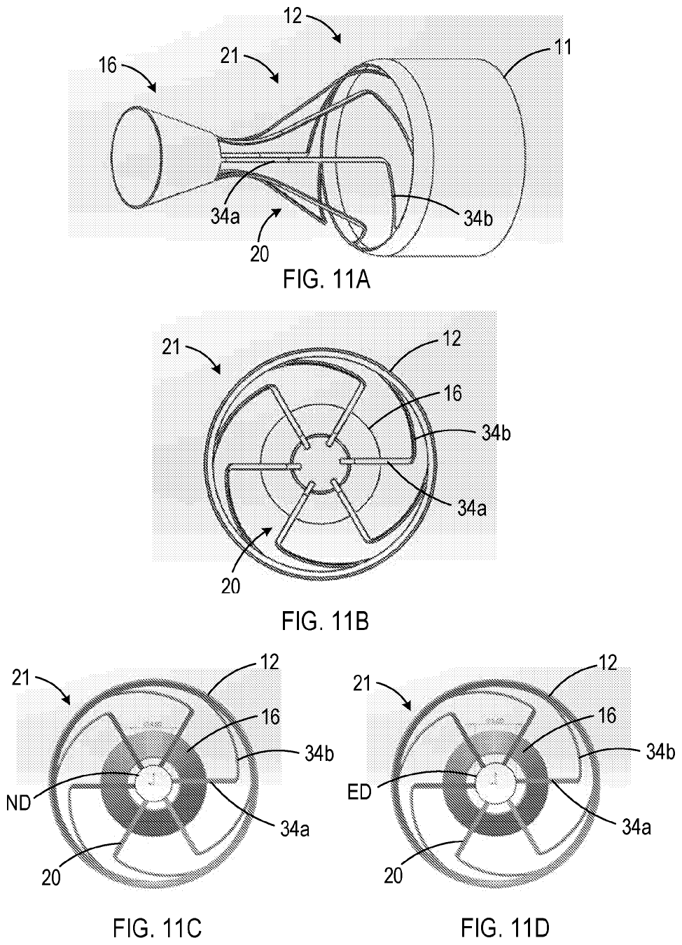

Referring now to FIGS. 10A to 10C, another exemplary expandable portion of the nozzle of the dynamic flow modulator is provided. As shown in FIGS. 10A and 10B, frame 20 forming flow modulator 20 may a first frame forming a portion of upstream component 12, e.g., cells 20b1, and a second frame forming downstream component 16, e.g., cells 20c, 20d, and 20e, such that there is no frame extending in section 20b2 between cells 20b1 and cells 20c. Moreover, both the first and second frames of frame 20 may be coated with biocompatible material 22, such that biocompatible material 22 extends continuously across section 20b2 from cells 20b1 to cells 20c, thereby forming a continuous fluid flow path through flow modulator 10. Accordingly, biocompatible material 22 extending across section 20b2 forms expandable portion 21 and is therefore radially expandable responsive to increased upstream pressures. FIG. 10C is a cross-sectional view of the nozzle portion along line 8C-8C of FIGS. 10A and 10B. As shown in FIG. 10C, biocompatible material 22 at expandable portion 21 may expand radially from nominal diameter ND towards expanded diameter ED.

Referring now to FIGS. 11A to 11D, another exemplary expandable portion of the nozzle of the dynamic flow modulator is provided. As shown in FIGS. 11A and 11B, the portion of frame 20 forming expandable portion 21 may be formed of a plurality of thin struts extending between upstream component 12 and downstream component 16. For example, each of the plurality of thin struts may include first straight portion 34a extending axially from downstream component 16 towards inlet 11 of upstream component 12, and second portion 34b extending circumferentially and axially from first straight portion 34a toward inlet 11 of upstream component 12. The bend at the junction between first straight portion 34a and second portion 34b permits expandable portion 21 to expand radially responsive to increased upstream pressures. For example, the increased upstream pressure will apply a force against second portion 34b, thereby causing expandable portion 21 to expand radially.

FIG. 11C illustrates the diameter of the nozzle at its nominal diameter ND, and FIG. 11D illustrates the diameter of the nozzle at its expanded diameter ED. By comparing FIGS. 11C and 11D, as expandable portion 21 expands radially, the distance between first straight portions 34a of frame 20 increases.

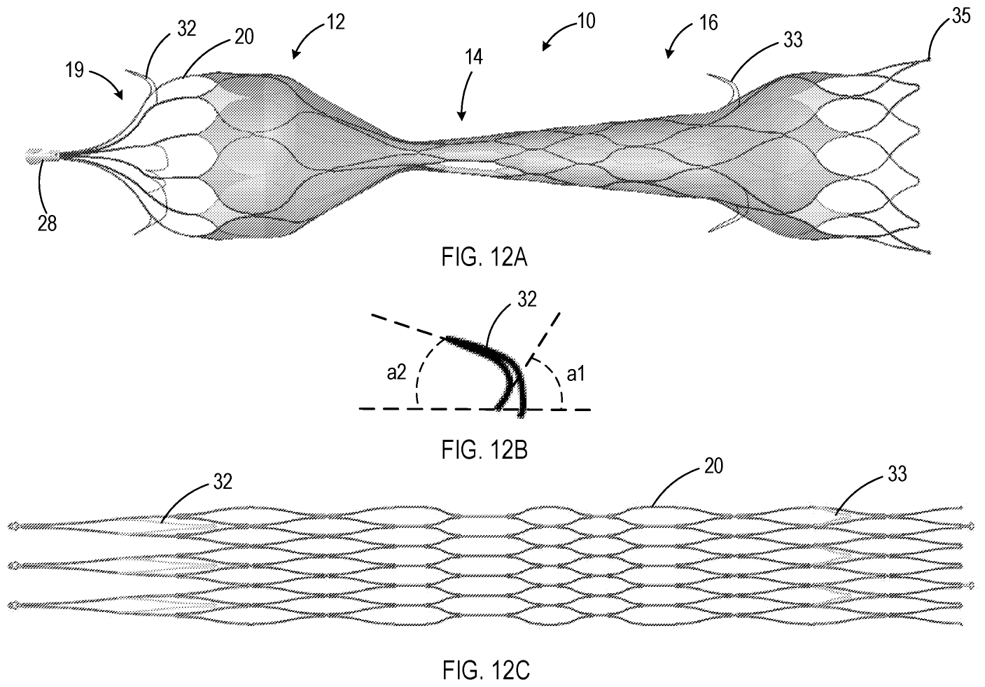

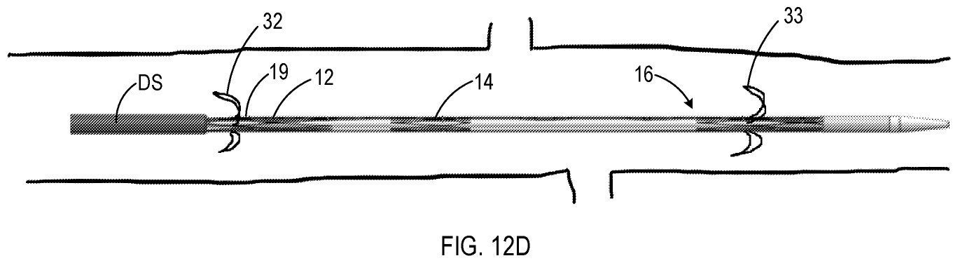

Referring now to FIGS. 12A to 12C, flow modulator 10 may include a plurality of fixation elements for securing flow modulator 10 within a vessel, and thereby prevent migration, e.g., upstream migration, of flow modulator 10 within the vessel. For example, as shown in FIG. 12A, retrieval portion 19 of flow modulator 10 may include a plurality of anchors 32 extending away from the external surface of frame 20. Anchors 32 may be disposed circumferentially along the outer surface of flow modulator 10, and may be transitionable from a collapsed delivery state to an expanded deployed state, as shown in FIG. 12A. Additionally, or alternatively, plurality of anchors 33 may be disposed on downstream component 16, e.g., at a location upstream of the sealing zone of downstream component 16, to further engage with the vessel.

FIG. 12B illustrates the curvature of each of the fixation elements, e.g., fixation elements 32 or 33. Illustratively, as shown in FIG. 12B, a first portion of fixation element 32 extends radially from the outer surface of frame 20 at a first angle a1 towards the downstream direction, and a second portion of fixation element 32 extends radially from the distal end of the first portion of fixation element 32 at a second angle a2 towards the upstream direction, thereby forming a bend. Preferably, angle a1 is larger than a2. As angle a2 is in the direction of the hook, the pre-formed bend permits sheathing of flow modulator 10 with a delivery sheath that advances from the distal side of flow modulator 10. The flexibility of fixation elements 32 and 33 is sufficiently high, such that they may be compressed radially by the vessel, e.g., IVC, yet if the vessel wall suddenly expands, e.g., due to coughing, fixation elements 32 and 33 return to their nominal, expanded diameter to thereby push against the vessel wall and prevent stent migration.

As shown in FIG. 12C, fixation elements 32 and 33 may lie in a straightened, flat configuration in the collapsed delivery state, where both the first and second portions of the fixation elements are substantially parallel to the longitudinal axis of flow modulator 10. Moreover, fixation elements 32 and 33 may be coupled to adjacent cells formed by frame 20, and/or may be coupled to the cells in a circumferentially spaced out pattern, e.g., every 2, 3, or 4 cells. For example, in FIG. 12C, fixation elements 33 are coupled to every other cell at downstream component 16.

Referring again to FIG. 12A, the distal ends of downstream component 16 may include additional fixation elements, e.g., barbs 35, that may penetrate the tissue to thereby prevent migration of flow modulator 10 within the vessel. For example, barbs 35 may be curved radially outward from the longitudinal axis of flow modulator 10.

As described above, flow modulator 10 may be delivered using a delivery system such as described in U.S. Pat. No. 11,324,619 to Yacoby. Accordingly, when flow modulator 10 is in a collapsed delivery state within a delivery sheath, fixation elements 32 and 33 extend parallel to the longitudinal axis of flow modulator 10 in a collapsed delivery state, such that the second portions of the fixation elements is downstream of the first portions of the respective fixation elements. FIG. 12D illustrates the unsheathing of flow modulator 10, prior to deployment of flow modulator 10. As shown in FIG. 12D, when delivery sheath DS is retracted to expose flow modulator 10, fixation elements 32 and 33 may self-deploy, e.g., curl/rotate to their preset configuration, such that the second portions of the respective fixation elements extend at angle a2 and the first portions of the respective fixation elements extend at angle a1 from flow modulator 10, without damaging the vessel wall. Flow modulator 10 may then be fully deployed within the vessel.

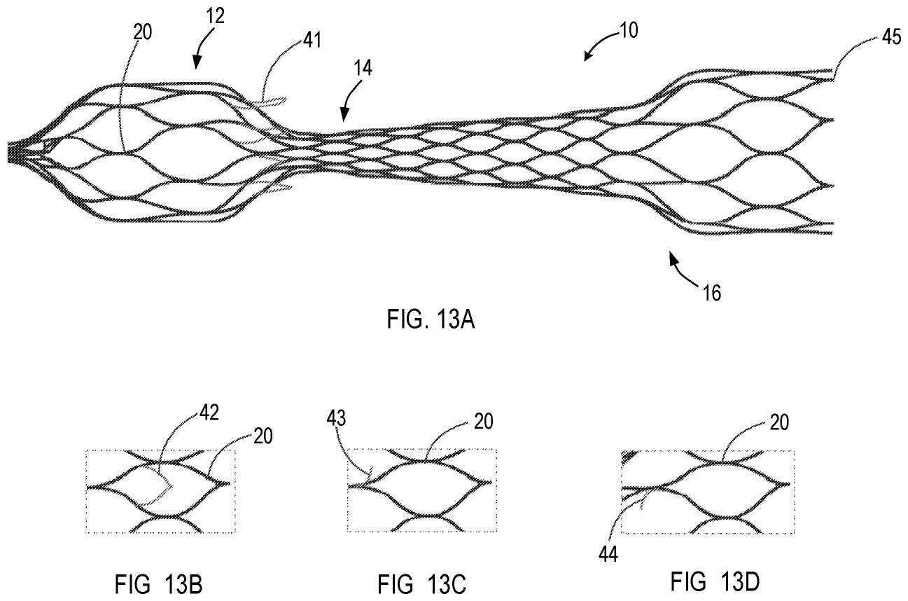

Referring now to FIGS. 13A to 13D, flow modulator 10 may include one or more various types of fixation elements for securing flow modulator 10 within a vessel, and thereby prevent migration of flow modulator 10 within the vessel. For example, as shown in FIG. 13A, upstream component 12 of flow modulator 10 may include a plurality of anchors 41 extending away from the external surface of frame 20, e.g., at a location downstream of the sealing zone of upstream component 12. Anchors 41 may be disposed circumferentially along the outer surface of flow modulator 10. Additionally, or alternatively, plurality of anchors 41 may be disposed on downstream component 16, to engage with the vessel. In addition, as shown in FIG. 13A, the distal ends of downstream component 16 may include additional fixation elements, e.g., barbs 45, that may penetrate the tissue to thereby prevent migration of flow modulator 10 within the vessel. For example, barbs 45 may be curved radially outward from the longitudinal axis of flow modulator 10.

As shown in FIG. 13B, the plurality of anchors extending from frame 20 may include anchors 42 with two ends coupled to frame 20, thereby forming a flap-like anchor. As shown in FIG. 13C, the plurality of anchors extending from frame 20 may include anchors 43, which extends upward from frame 20. As shown in FIG. 13D, the plurality of anchors extending from frame 20 may include anchors 44, which extends downward from frame 20. Anchors 41, 42, 43, 44 may engage with the vessel to thereby prevent migration of flow modulator 10. In some embodiments, anchors 41, 42, 43, 44 may penetrate the tissue to secure flow modulator 10 within the vessel.

Moreover, the flow modulators described herein may be used in conjunction with an external pump and a control system as described in WO 2020/109979, the entire contents of which are incorporated herein by reference. For example, the external pump may be an intermittent pneumatic compression (IPC) or a cardiac enhanced external counter-pulsation (EECP) pump (such as the ArtAssist® device, available by ACI Medical, San Marcos, California). The pump may be programmed to mimic the natural pumping action of an ambulatory calf and/or foot to move blood in the deep veins of the leg, thereby reducing deep vein thrombosis formation. In addition, the pump may provide power to the flow modulator. The external pump and the control system may be fully mobile and/or battery operated. For example, the external pump and the control system be worn by the patient, e.g., around the patient's leg.

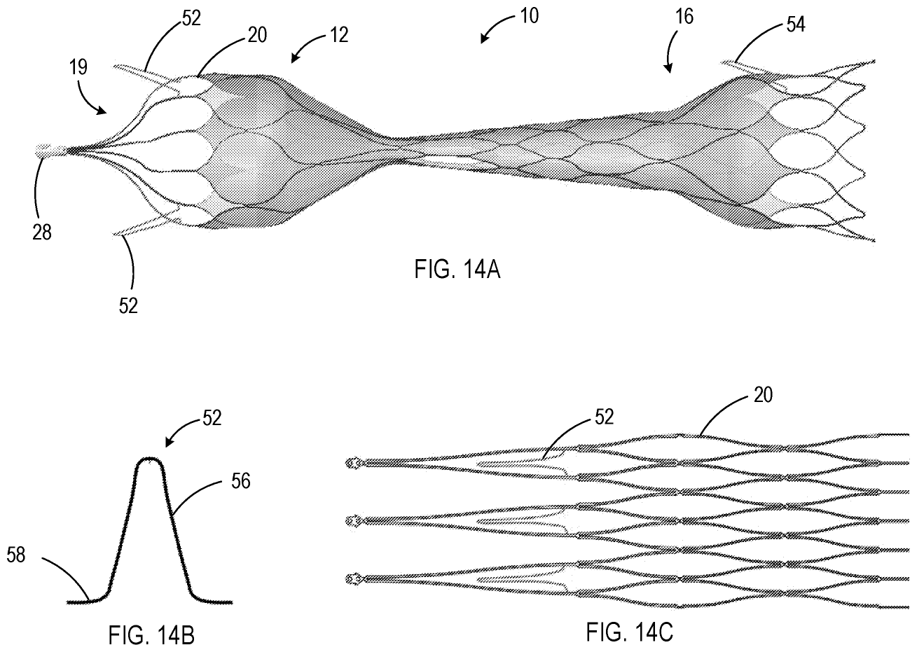

Referring now to FIGS. 14A to 14C, flow modulator 10 may include one or more various types of fixation elements for securing flow modulator 10 within a vessel, and thereby prevent migration of flow modulator 10 within the vessel. For example, at least one anchor 52 may extend outwardly from the surface of frame 20 to secure flow modulator 10. As shown in FIG. 14A, anchors 52 may extend outwardly from upstream component 12 in an upstream, i.e., proximal, direction towards retrieval portion 19. Moreover, anchor 52 may extend outwardly at an angle to facilitate engagement with the vessel wall.

As shown in FIG. 14B, each anchor 52 may include attachment portion 58 for coupling anchor 52 to frame 20, and anchor portion 56 extending from attachment portion 58. Attachment portion 58 may attach anchor 52 to frame 20 in a hinge-like manner, such that anchor portion 56 is rotatable with respect to frame 20 about attachment point 58, e.g., upon application of a force against anchors 52 in the downstream direction during retrieval of flow modulator 10 via retrieval portion 19. Moreover, anchors 52 may be formed of an elastic material, e.g., Nitinol, such that during retrieval of flow modulator 10, anchors 52 may plastically deform upon rotation about frame 20. For example, anchors 52 may be permanently plastically deformed upon being flipped relative to frame 20 during retrieval of flow modulator 10, such that flow modulator 20 must be removed and cannot be repositioned due to the plastic deformation of anchors 52.

As shown in FIG. 14B, anchor portion 56 may be substantially triangular. Accordingly, anchor portion 56 may engage with the vessel to thereby prevent migration of the flow modulator 10. Additionally, or alternatively, a plurality of anchors 54 may be disposed on downstream component 16, to engage with the vessel. Anchors 54 may be constructed similar to anchors 52 with an attachment portion coupled to frame 20 and an anchor portion extending from the attachment portion, such that anchors 54 also may extend outwardly from downstream component 16 in an upstream, i.e., proximal, direction, and may be rotated about the attachment portion upon application of a force against anchor 54 in the downstream direction. For example, during retrieval of flow modulator 10, a sheath may be advanced over hook 28 and retrieval portion 19 and cause anchors 52 to rotate from an upstream facing direction to a downstream facing direction, such that upstream component 12 collapses into the sheath. In addition, the sheath may be further advanced distally toward downstream component 16 and cause anchors 54 to rotate from an upstream facing direction to a downstream facing direction, such that downstream component 16 collapses into the sheath. Anchors 52 and anchors 54 may be of similar length and attached at a similar angle to frame 20.

In some embodiments as described above where the flow modulator includes a distal retrieval portion, e.g., retrieval portion 31, anchors 54 may extend outwardly from downstream component 16 in a downstream, i.e., distal, direction towards retrieval portion 31. Accordingly, anchors 54 may be rotated about the attachment portion upon application of a force against anchors 54 in the upstream direction during retrieval of flow modulator 10 via retrieval portion 31.

As shown in FIG. 14C, anchors 52 may be disposed along the outer surface of frame 20 in a manner such that anchors 52 are disposed circumferentially along the outer surface of flow modulator 10. In some embodiments, flow modulator 10 may include the same number of anchors and cells, such that anchors 52 are symmetrically distributed about the circumference of flow modulator 10. For example, as illustrated in FIG. 14C, frame 20 may include three cells at the upstream end of frame 20, and accordingly may include three anchors 52. In other embodiments, flow modulator 10 may include fewer anchors than cells. For example, flow modulator 10 may include two, three, or four anchors 52 where flow modulator 10 has six cells. Alternatively, flow modulator 10 may include more anchors that cells at the upstream end of frame 20. For example, when the upstream ends of the three cells of frame 20 converge together, the space formed between each of the three cells may accommodate an anchor.

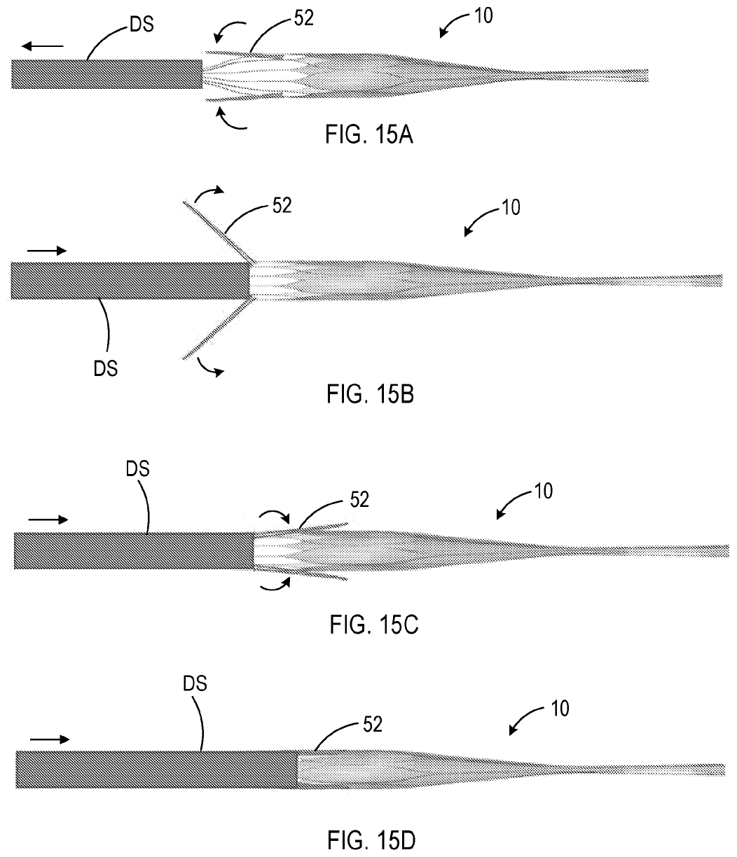

Referring now to FIGS. 15A to 15D, flow modulator 10 may be deployed and retrieved using delivery sheath DS. Flow modulator 10 may be fully contained within delivery sheath DS prior to placement within the vessel so that flow modulator 10 may be delivered to and deployed at the desired location within the vessel. When the distal end of delivery sheath DS is positioned at the desired deployment location, delivery sheath DS may be retracted, e.g., pulled in the upstream direction while flow modulator 10 remains stationary relative to the vessel. As illustrated in FIG. 15A, retraction of delivery sheath DS may expose flow modulator 10, having anchors 52 extending outwardly from frame 20 in the upstream direction. Anchors 52 may be biased to extend outwardly from frame 20 in the upstream direction upon removal from delivery sheath DS to facilitate securing flow modulator 10 within the vessel. When fully deployed, flow modulator 10 may resemble the embodiment illustrated in FIG. 14A.

When removal/retrieval of flow modulator 10 is desired, delivery sheath DS may be advanced, e.g., pushed in a downstream direction over flow modulator 10, to thereby collapse flow modulator 10 within delivery sheath DS for removal. As illustrated in FIG. 15B, as delivery sheath DS is advanced over flow modulator 10, delivery sheath DS may contact anchors 52 at the point where anchors 52 are attached to frame 20, e.g., the attachment portion of anchors 52. Further advancement of delivery sheath DS may force anchors 52 to rotate towards the downstream direction, expanding the angle between anchors 52 and the longitudinal axis of frame 20, as shown in FIG. 15C. After anchors 52 have flipped to extend in the downstream direction, advancement of delivery sheath DS may reduce the angle between anchors 52 and frame 20 while anchors 52 extend in the downstream direction. Reduction of the angle between anchors 52 and frame 20 may enable anchors 52 to collapse into delivery sheath DS as it is advanced over flow modulator 10, as shown in FIG. 15D. As described above, anchors 52 may be plastically deformed, e.g., permanently plastically deformed, upon being flipped relative to frame 20 during retrieval of flow modulator 10.

When flow modulator 10 is again collapsed within delivery sheath DS, delivery sheath DS containing flow modulator 10 may be repositioned within the vessel or removed from the vessel. As described above, flow modulator 10 may have additional anchors 54 disposed at the downstream portion of flow modulator 10. Accordingly, delivery sheath DS may be retracted to deploy anchors 54 in the upstream direction, and advanced over the flow modulator to flip anchors 54 and collapse the flow modulator within delivery sheath DS for retrieval in the same manner described above with regard to anchors 52.

As described above, in some embodiments, flow modulator 10 may include anchors disposed at the downstream component that are biased toward extending in the downstream direction. Accordingly, during delivery of the flow modulator, delivery sheath DS may interact with the downstream end of flow modulator 10, where delivery sheath DS may be retracted, e.g., pulled in the downstream direction, to expose flow modulator 10 with anchors 54 extending outwardly from frame 20 in the downstream direction. To facilitate removal of flow modulator 10, delivery sheath DS may be advanced, e.g., pushed in an upstream direction, to contain flow modulator 10. While being advanced, delivery sheath DS may contact and force anchors 54 to rotate towards the upstream direction. After anchors 54 have flipped to extend in the upstream direction, anchors 54 may collapse into delivery sheath DS as it is advanced over flow modulator 10. When flow modulator 10 is again collapsed within delivery sheath DS, delivery sheath DS containing flow modulator 10 may be repositioned within the vessel or removed from the vessel.

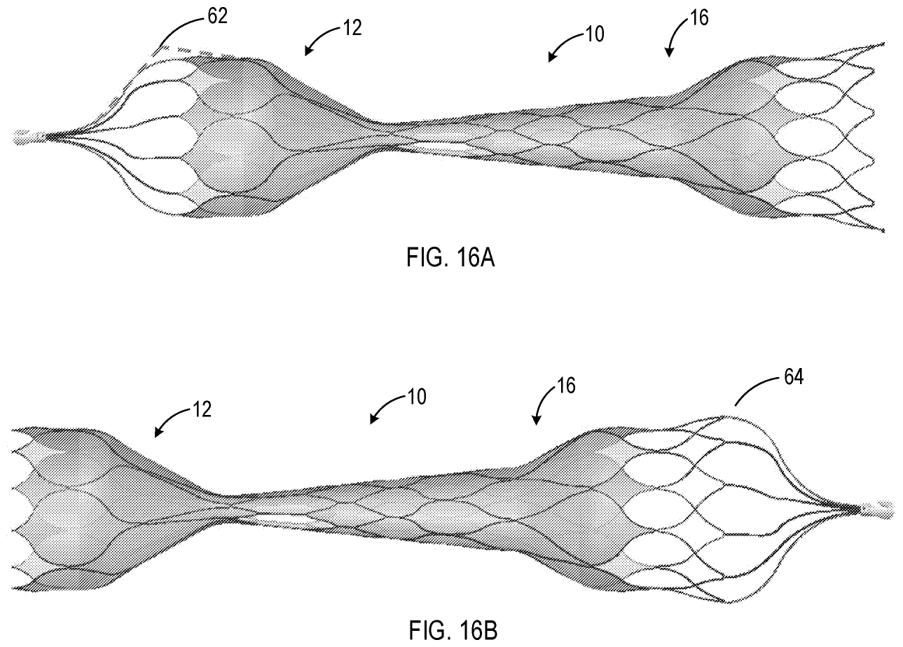

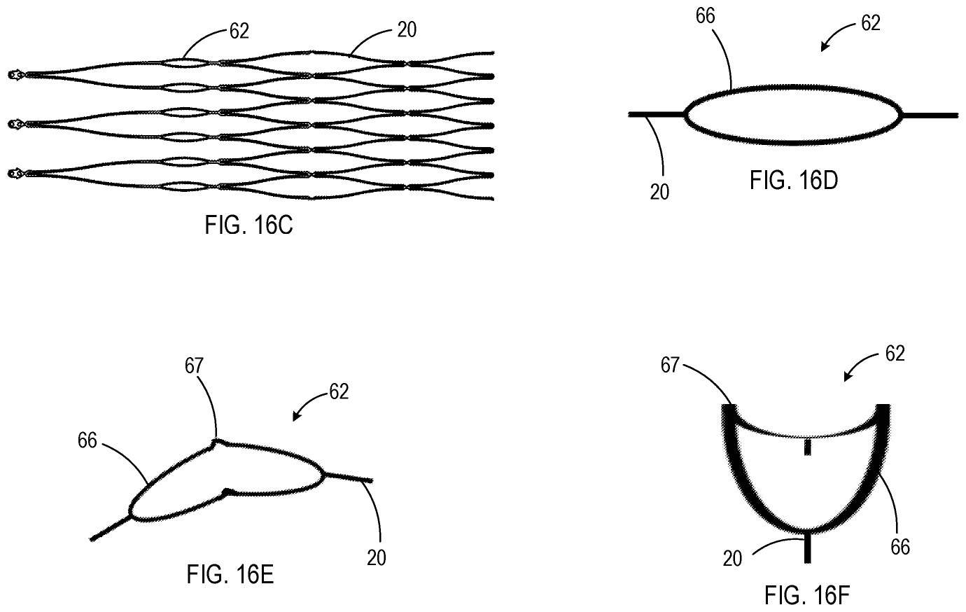

Referring now to FIGS. 16A to 16F, alternative fixation elements are provided. Specifically, flow modulator 10 may include one or more various types of fixation elements for securing flow modulator 10 within a vessel, and thereby prevent migration of flow modulator 10 within the vessel. For example, at least one anchor, e.g., anchors 62 and/or anchors 64, formed from frame 20 may extend outwardly from the surface of frame 20 to secure flow modulator 10. In some embodiments, as illustrated in FIG. 16A, anchors 62 extend outwardly from upstream component 12 in an upstream direction toward retrieval portion 19. In other embodiments, as illustrated in FIG. 16B, anchors 64 extend outwardly from downstream component 16 in a downstream direction toward retrieval portion 31. As would be understood by one of ordinary skill in the art, anchors 60 may be present on either or both of upstream component 12 or downstream component 16.

Anchors 62 and anchors 64 may be formed from a single frame structure 20. For example, as shown in FIG. 16C, which illustrates the downstream portion of frame 20 cut and flattened, frame 20 may include one or more cells formed therein, which may be bent to form anchors 62. For example, as shown in FIG. 16D, frame 20 may include one or more cells 66. Cells 66 may be, e.g., oval-shaped. During manufacturing, after frame 20 is cut, e.g., laser cut, the shape of frame 20 may be set so that frame 20 is properly shaped. During the shape-setting process, cells 66 may be bent upwards to create anchor points 67, e.g., protrusion-like bends, as shown in FIGS. 16E and 16F. Anchor points 67 may be effective to secure flow modulator 10 within the vessel when flow modulator is in its expanded state.

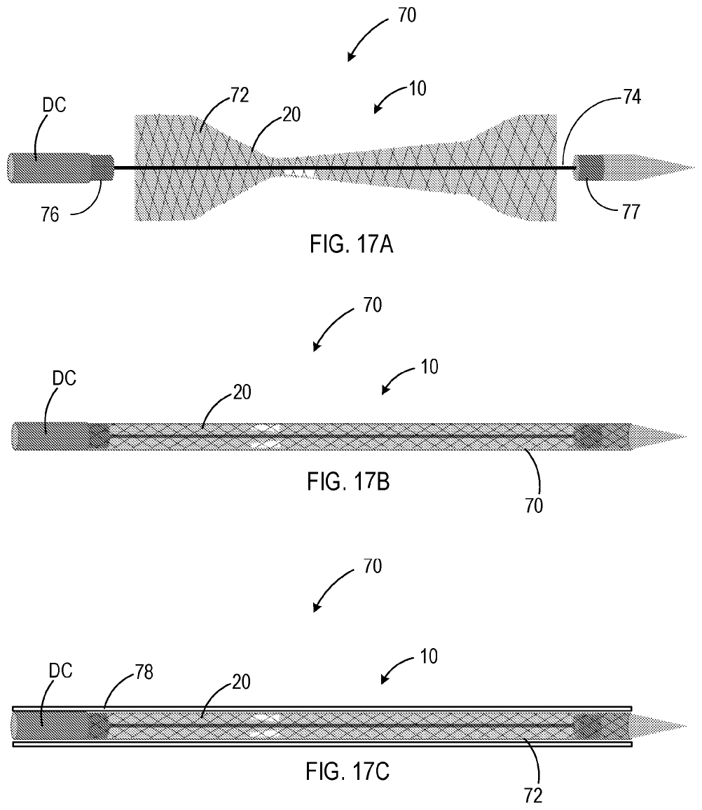

Referring now to FIGS. 17A to 17C, an alternative flow modulator 10 may be delivered to and placed within the vessel using delivery system 70. As shown in FIG. 17A, flow modulator 10 may include a braided frame 20. In some embodiments, frame 20 of flow modulator 10 may be covered in polymeric coating 72. Flow modulator 10 may be disposed over delivery shaft 74 attached to the distal end of delivery catheter DC. To load flow modulator 10 onto delivery system 70 for insertion through the patient's vasculature, flow modulator 10 may be collapsed and each end of flow modulator 10 may be fixed to upstream stent holder 76 and downstream stent holder 77, respectively. When flow modulator 10 is fixed to stent holders 76, 77 in the collapsed state, the profile of flow modulator 10 may be reduced to that of delivery catheter DC, as shown in FIG. 17B. During delivery, flow modulator 10 may remain exposed, e.g., without the need for a delivery sheath. In some embodiments, as shown in FIG. 17C, delivery system 70 may be contained within a thin sheath, e.g., sheath 78, such that flow modulator 10 is not exposed during delivery, e.g., to ease placement or repositioning within the vessel.

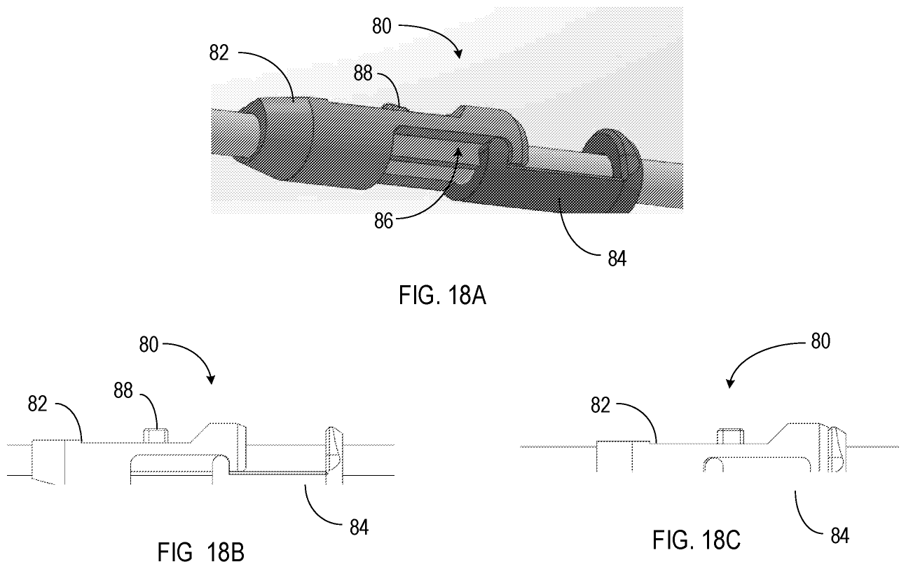

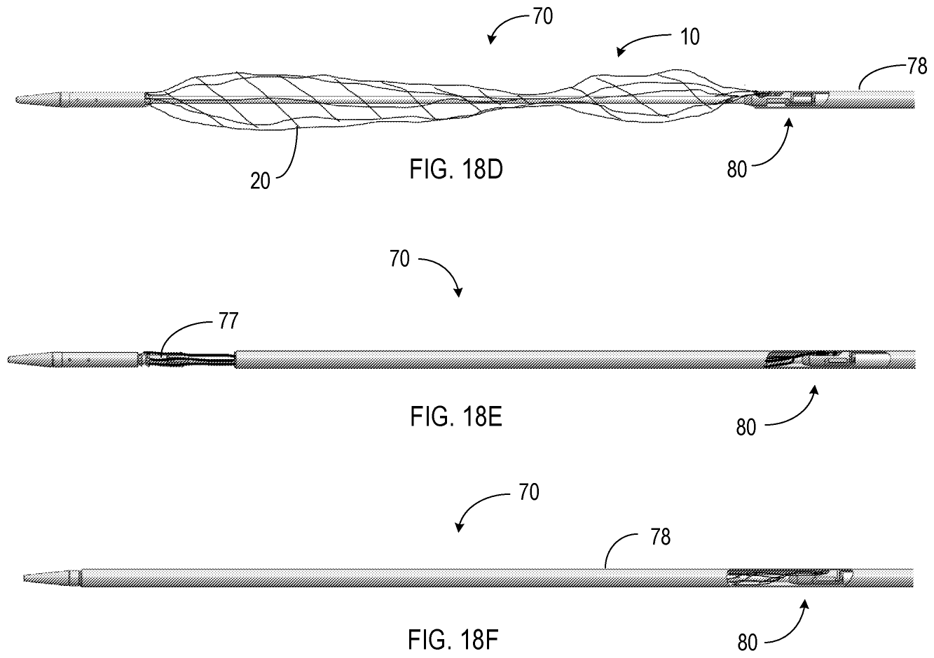

Referring now to FIGS. 18A to 18F, a stent holder that compensates for device shortening during deployment is provided. As shown in FIG. 18A, delivery system 70 may include a sliding stent holder 80, e.g., instead of upstream stent holder 76. Sliding stent holder 80 may include stationary component 84, which may be fixed to delivery system 70, and sliding component 82, which may be passively slidably coupled to stationary component 84 via slot connection 86. Accordingly, sliding component 82 may move axially relative to stationary component 84 along slot connection 86, e.g., responsive to tension applied by flow modulator 10 during deployment, such that sliding component 82 slides freely between the open and closed configurations during insertion and/or repositioning. In addition, sliding component 82 also may include knob 88 configured to removably attach to flow modulator 10.

Sliding component 82 of sliding stent holder 80 may slide along slot connection 86, alternating between an open configuration as shown in FIG. 18B and a closed configuration as shown in FIG. 18C. As shown in FIG. 18D, flow modulator 10 may be fixed to knob 88 and downstream holder 77 such that flow modulator 10 has a reduced profile while loaded onto delivery system 70. When sheath 78 is retracted such that flow modulator 10 is exposed, as shown in FIG. 18D, flow modulator 10 is biased toward the expanded state such that the length of flow modulator 10 shortens in the expanded state. In the expanded state, flow modulator 10 applies tension to sliding component 82 to thereby cause sliding component 82 to move distally relative to stationary component 84 toward the open configuration.

To prepare delivery system 70 and flow modulator 10 for delivery, a delivery sheath, e.g., delivery sheath 78 may be advanced over delivery system 70 and flow modulator 10 such that flow modulator 10 collapses within sheath 78, as shown in FIG. 18E. As flow modulator 10 collapses, the length of flow modulator 10 increases in the collapsed state, which applies tension to sliding component 82 to thereby cause sliding component 82 to move proximally relative to stationary component 84 toward the closed configuration, as shown in FIG. 18F. While in the collapsed state within sheath 78, sliding stent holder 80 permits flow modulator 10 to minimally shorten in length within sheath via sliding component 82, to thereby relieve tension of flow modulator 10 within sheath 78.

While preferred illustrative embodiments of the invention are described above, it will be apparent to one skilled in the art that various changes and modifications may be made therein without departing from the invention. For example, the flow modulators described herein may be installed in the inferior vena cava at the branch to a hepatic vein. Accordingly, additional blood may be entrained from the hepatic veins into the IVC, thereby improving splanchnic circulation. Acutely or chronically implanting a flow modulator in the IVC adjacent the hepatic veins may improve liver function and/or may be used instead of, or in parallel to, a TIPS procedure. Advantageously, the flow modulator improves hepatic flow to the inferior vena cava allowing blood to enter the liver for natural filtering (in contrast to a TIPS procedure that bypasses blood from the liver). The flow modulator, whether used together with a TIPS procedure or in place of a TIPS procedure, is expected to treat conditions such as portal hypertension (often due to liver cirrhosis) which frequently leads to intestinal bleeding, life-threatening esophageal bleeding (esophageal varices), the buildup of fluid within the abdomen (ascites), and/or hepatorenal syndrome.

Additionally or alternatively, the flow modulators described herein may be installed in the inferior vena cava to entrain additional blood from both the renal and hepatic veins. For example, the exit of the downstream component may be downstream to the hepatic vein while the inlet of the upstream component is upstream to the renal veins. In one study, the mean distance from a downstream renal vein to the hepatic vein was 6 cm, and the mean distance from the upstream-most renal vein to the downstream-most renal vein was 2.5 cm, and thus a flow modulator having an overall distance of 8.5 cm between the fixation areas of upstream component 12 and downstream component 16 may be anchored within the IVC to improve both renal and hepatic perfusion simultaneously.

Moreover, the flow modulators described herein may be installed in an aneurysm to lower pressure at the aneurysm site, and reduce the risk that the aneurysm will increase in size or burst, and may even cause the aneurysm to decrease in size. In this case, the flow modulator is expected to provide beneficial effect even without sealing against the aneurysm. In addition, if there are one or more side branch lumens at or near the aneurysm site, the device not only will reduce the pressure but also permit blood to flow to the side branches. In this application, the device of the present invention provides significant benefit as compared to previously-known circular stent grafts, which disadvantageously may block the side branches. If there are no side branches, then the device is expected to reduce pressure without increasing the blood flow. Optionally, a filter may be used with the flow modulator to prevent embolic debris from flowing from the aneurysm to other blood vessels.

Any of the foregoing embodiments of the device of the present invention may serve to divert emboli or other debris, so there is no need to use an extra filtration device. One example is using the upstream component or downstream component at or near the carotid arteries to divert emboli or other debris.

While preferred illustrative embodiments of the invention are described above, it will be apparent to one skilled in the art that various changes and modifications may be made therein without departing from the invention. The appended claims are intended to cover all such changes and modifications that fall within the true spirit and scope of the invention.

1. A flow modulator device for altering fluid flow through a body lumen, the body lumen coupled to a branch lumen, the flow modulator device comprising:
a stent configured to be positioned within the body lumen, the stent comprising an upstream component having an inlet, an outlet, and a cross-sectional flow area that converges from the inlet towards the outlet to form a nozzle, at least a portion of the nozzle configured to be radially expandable responsive to pressure upstream of the flow modulator device within the body lumen, a downstream component having an entry, an exit, and a cross-sectional flow area that diverges from the entry towards the exit, and an entrainment region between the inlet of the upstream component and the exit of the downstream component, the entrainment region comprising one or more openings,
wherein the nozzle is configured to accelerate a fluid stream passing through the upstream component towards the downstream component to generate a low pressure region in a vicinity of the entrainment region that entrains additional fluid into the fluid stream via the one or more openings as the fluid stream passes into the downstream component.

2. The flow modulator device of claim 1, wherein the upstream component and the downstream component are formed from a frame, and are at least partially coated with a biocompatible material, thereby exposing the one or more openings and defining the inlet.

3. The flow modulator device of claim 2, wherein the radially expandable portion of the nozzle is formed by a continuous passageway of biocompatible material extending through the flow modulator device within an uncoated portion of the frame, the continuous passageway of biocompatible material having an outer diameter less than an inner diameter of the uncoated portion of the frame, such that the continuous passageway of biocompatible material radially expands toward the uncoated portion of the frame responsive to pressure upstream of the flow modulator device within the body lumen.

4. The flow modulator device of claim 2, wherein the radially expandable portion of the nozzle is formed by a coated portion of the frame having more flexibility than other portions of the frame, such that it radially expands responsive to pressure upstream of the flow modulator device within the body lumen.

5. The flow modulator device of claim 4, wherein the coated portion of the frame having more flexibility than other portions of the frame comprises thin Nitinol wires.

6. The flow modulator device of claim 4, wherein the biocompatible material coating the portion of the frame having more flexibility than other portions of the frame is configured to radially expand beyond an outer diameter of the frame responsive to pressure upstream of the flow modulator device within the body lumen.

7. The flow modulator device of claim 2, wherein the radially expandable portion of the nozzle is formed by a continuous passageway of biocompatible material extending along a frameless portion of the flow modulator device between the frame forming at least a portion of the upstream component and the frame forming the downstream component, such that the continuous passageway of biocompatible material radially expands responsive to pressure upstream of the flow modulator device within the body lumen.

8. (canceled)

9. The flow modulator device of claim 2, wherein the frame defines a plurality of cells.

10. The flow modulator device of claim 9, wherein at least some cells of the plurality of cells adjacent to at least one of the inlet of the upstream component or the exit of the downstream component comprises an oval shape, the at least some cells comprising a preformed bend configured to extend outwardly to secure the flow modulator device within the body lumen.

11. The flow modulator device of claim 2, wherein at least a portion of a distal portion of the downstream component is not coated with the biocompatible material, the distal portion configured to adapt with the body lumen to thereby prevent migration of the flow modulator device within the body lumen.

12. The flow modulator device of claim 1, wherein the radially expandable portion of the nozzle is configured to radially expand responsive to pressure upstream of the flow modulator device within the body lumen without causing expansion of portions of the upstream and downstream components that engage the body lumen.

13. The flow modulator device of claim 1, wherein the stent is configured to transition from a collapsed delivery state to an expanded deployed state within the body lumen.

14. The flow modulator device of claim 1, wherein the one or more openings comprises a plurality of openings radially spaced around the entrainment region.

15. The flow modulator device of claim 1, wherein at least one of the upstream component or the downstream component comprises a plurality of fixation elements extending outwardly therefrom to secure the flow modulator device within the body lumen.

16-20. (canceled)

21. The flow modulator device of claim 1, wherein the upstream component comprises a retrieval portion configured to facilitate retrieval of the flow modulator.

22-24. (canceled)

25. The flow modulator device of claim 1, wherein the downstream component comprises a first diverging portion and a second diverging portion downstream from the first diverging portion, the second diverging portion's average angle of divergence greater than the first diverging portion's average angle of divergence.

26. The flow modulator device of claim 1, wherein the downstream component comprises a downstream retrieval portion configured to facilitate retrieval of the flow modulator.

27-29. (canceled)

30. The flow modulator device of claim 1, wherein the downstream component comprises a diffuser that decelerates the fluid stream having the entrained additional fluid passing through the downstream component.

31. The flow modulator device of claim 1, wherein the entrainment region is integrally formed with the downstream component.

32. A delivery system configured to deliver the flow modulator device of claim 1, the delivery system comprising:
a delivery catheter comprising one or more holders configured to be releasably coupled to the flow modulator device in a collapsed delivery state.

33. The delivery system of claim 32, further comprising:
a sheath configured to be disposed over the delivery catheter and flow modulator device to maintain the flow modulator in the collapsed delivery state,
wherein, upon retraction of the sheath relative to the delivery catheter to expose the flow modulator device, the flow modulator device transitions to an expanded deployed state.

34-41.
